(19) United States
(12) Patent Application Publication
(10) Pub. No.: US 2009/0137527 A1
(43) Pub. Date: May 28, 2009

(54) Compositions and methods for modulating immune function

(75) Inventors: Per Gjorstrup, Cambridge, MA (US); Shixin Qin, Lexington, MA (US); Lijun Wu, Lexington, MA (US)

Correspondence Address: ROPES & GRAY LLP, PATENT DOCKETING 39/41, ONE INTERNATIONAL PLACE, BOSTON, MA, 02110-2624, US

(73) Assignee: Resolvyx Pharmaceuticals, Inc., Bedford, MA (US)

(21) Appl. No.: 12/283,616

(22) Filed: Sep. 12, 2008

Related U.S. Application Data
(60) Provisional application No. 60/993,774, filed on Sep. 14, 2007.

(51) Int. Cl. A61K 31/695; A61K 31/232; A61K 31/202; A61K 31/60; A61P 37/02

(52) U.S. Cl. 514/63; 514/549; 514/560; 514/165

(57) ABSTRACT

The invention relates to methods of modulating immune function, suppressing immune response, treating autoimmune diseases or autoimmune disorders, and treating diseases, sequelae or pathological conditions mediated by an activation of the immune system comprising administering a compound of formula A, a compound of any one of formulae 1-49, a lipoxin compound, or an oxylipin compound.

### RELATED APPLICATIONS

This application claims the benefit of priority to U.S. Provisional Patent Application No. 60/993,774, filed Sep. 14, 2007, which application is hereby incorporated by reference in its entirety.

### BACKGROUND

The immune system is a complicated network of cells and cell components (molecules) that normally work to defend the body and eliminate infections caused by bacteria, viruses, and other invading foreign bodies. If a person has an autoimmune disease, the immune system mistakenly attacks itself, targeting the cells, tissues and organs of a person's own body. Some autoimmune diseases are known to begin or worsen with certain triggers such as viral, parasitic and chronic bacterial infections. Other less-understood influences that affect the immune system and the course of autoimmune diseases include aging, chronic stress, hormones and pregnancy. There are many different autoimmune diseases, and they can each affect the body in different ways. Many of the autoimmune diseases are rare; however, as a group, autoimmune diseases afflict millions of people.

Autoimmune diseases are often chronic, requiring lifelong care and monitoring, even when the person may look or feel well. Currently, few autoimmune diseases can be cured or made to go into remission with treatment. Physicians most often help patients manage the consequences of inflammation caused by the autoimmune disease. In some people, a limited number of immuno-suppressive medications may result in disease remission. However, even if their disease goes into remission, patients are rarely able to discontinue medication, and the long-term side effects of immunosuppressive medication can be substantial.

Immunomodulators are useful for treating systemic autoimmune diseases, such as lupus erythematosus and diabetes, as well as immunodeficiency diseases. Immunomodulators are also useful for immunotherapy of cancer or to prevent rejections of foreign organs or other tissues in transplants, e.g., kidney, heart, or bone marrow. Examples of immunomodulators include FK506, muramylic acid dipeptide derivatives, levamisole, niridazole, oxysuran, flagyl, and others from the groups of interferons, interleukins, leukotrienes, corticosteroids, and cyclosporins. Many of these compounds, however, have undesirable side effects and/or high toxicity in a subject in need thereof. As such, there remains a need for additional treatments.

### SUMMARY OF INVENTION

The present invention provides a method of inhibiting immune function in a patient, comprising administering to said patient a compound of formula A, a compound of any one of formulae 1-49, a lipoxin compound, an oxylipin compound, or a combination of aspirin and an omega-3 fatty acid.

The present invention provides a method of suppressing an immune response in a patient, comprising administering to said patient a compound of formula A, a compound of any one of formulae 1-49, a lipoxin compound, an oxylipin compound, or a combination of aspirin and an omega-3 fatty acid.

The present invention provides a method of treating an autoimmune disease or an autoimmune disorder in a patient, comprising administering to said patient a compound of formula A, a compound of any one of formulae 1-49, a lipoxin compound, an oxylipin compound, or a combination of aspirin and an omega-3 fatty acid.

The present invention further provides a method of treating a disease, sequela or pathological condition mediated by an activation of the immune system in a patient, comprising administering to said patient a compound of formula A, a compound of any one of formulae 1-49, a lipoxin compound, an oxylipin compound, or a combination of aspirin and an omega-3 fatty acid.

### DETAILED DESCRIPTION OF THE DRAWINGS

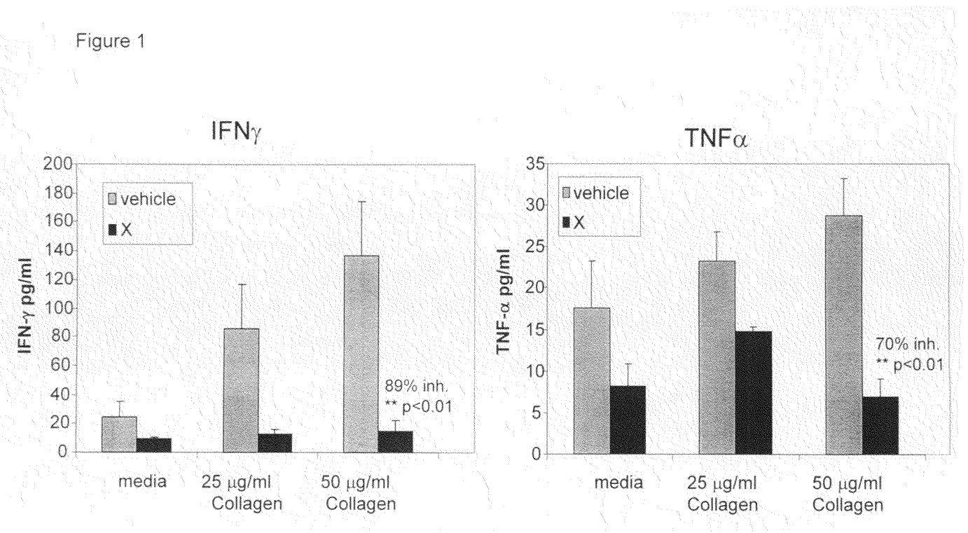

FIG. 1 shows that Compound X inhibited ex vivo IFN-γ and TNFα production in lymph node cells from collagen-induced arthritis rats.

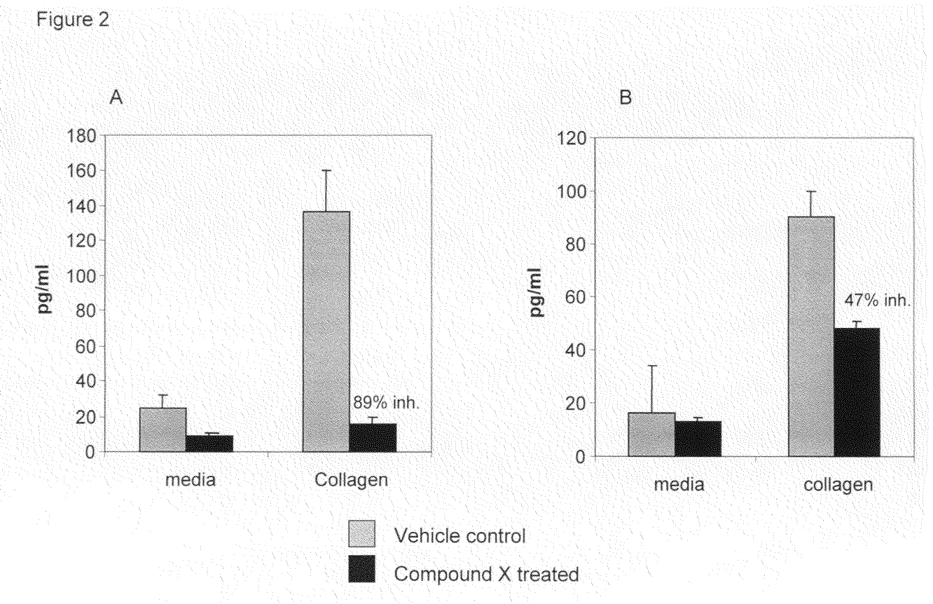

FIGS. 2a and 2b show that Compound X inhibited ex vivo collagen-induced IFN-γ production in lymph node cells from collagen-induced arthritis rats using two different treatment regimens.

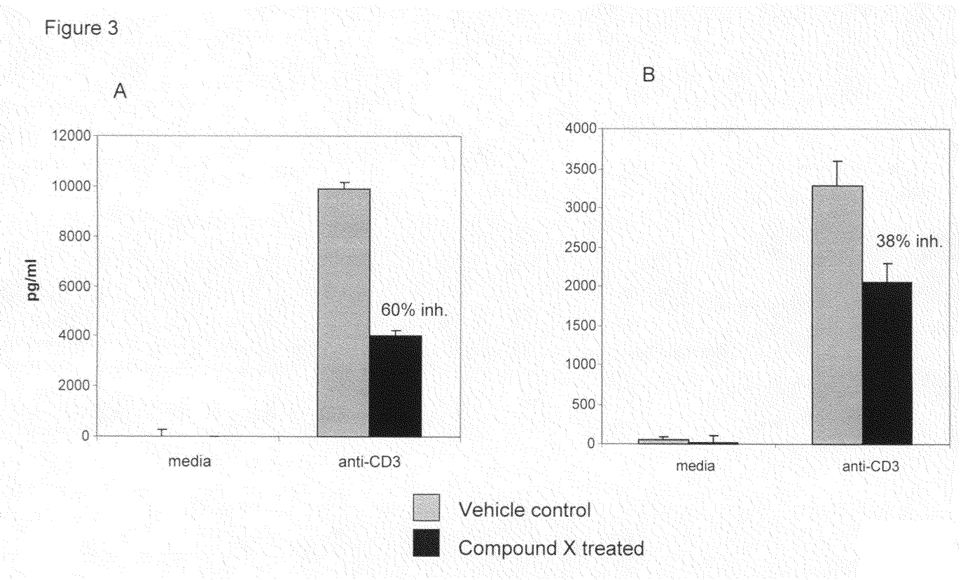

FIGS. 3a and 3b show that Compound X inhibited ex vivo anti-CD3 mAb-induced IL-17 production in lymph node cells from collagen-induced arthritis rats using two different treatment regimens.

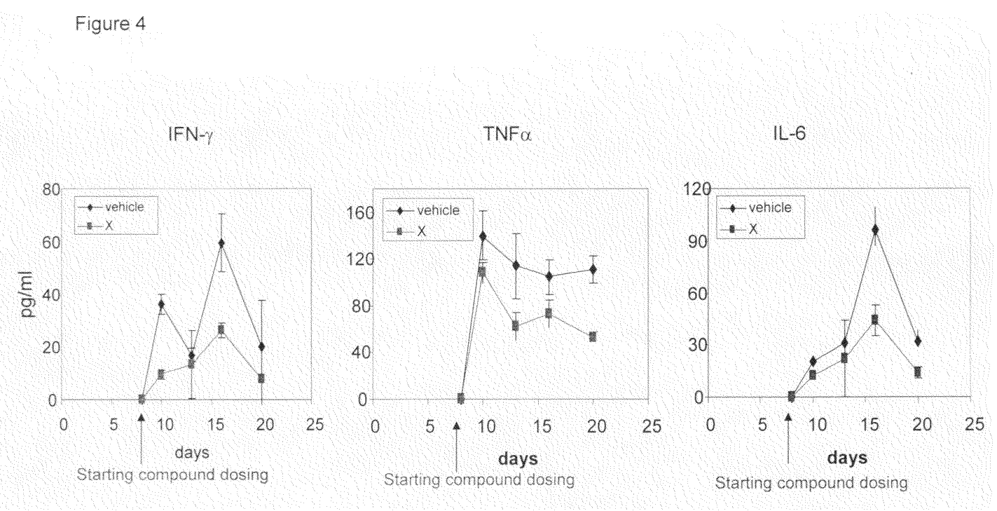

FIG. 4 shows that Compound X inhibited ex vivo LPS-stimulated cytokines in whole blood from CIA rats.

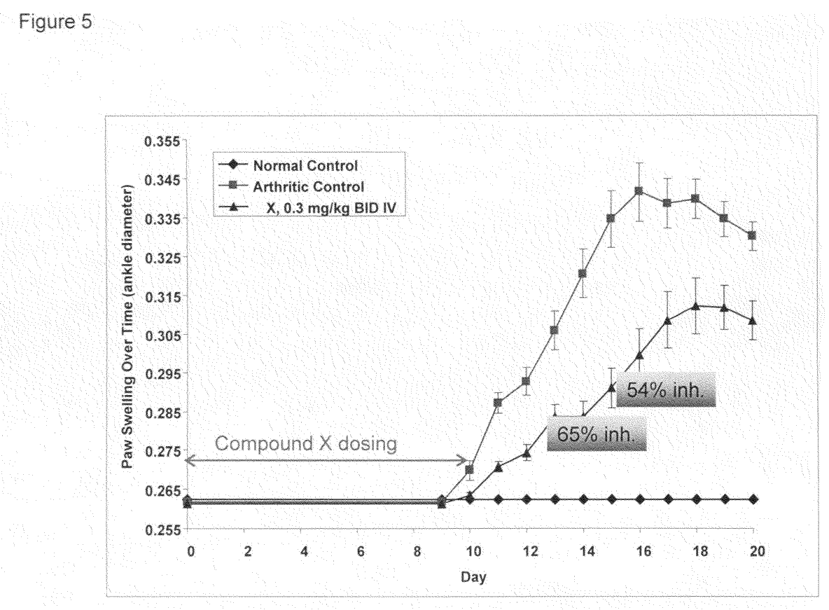

FIG. 5 shows that prophylactic dosing of Compound X inhibited arthritis in rats with CIA.

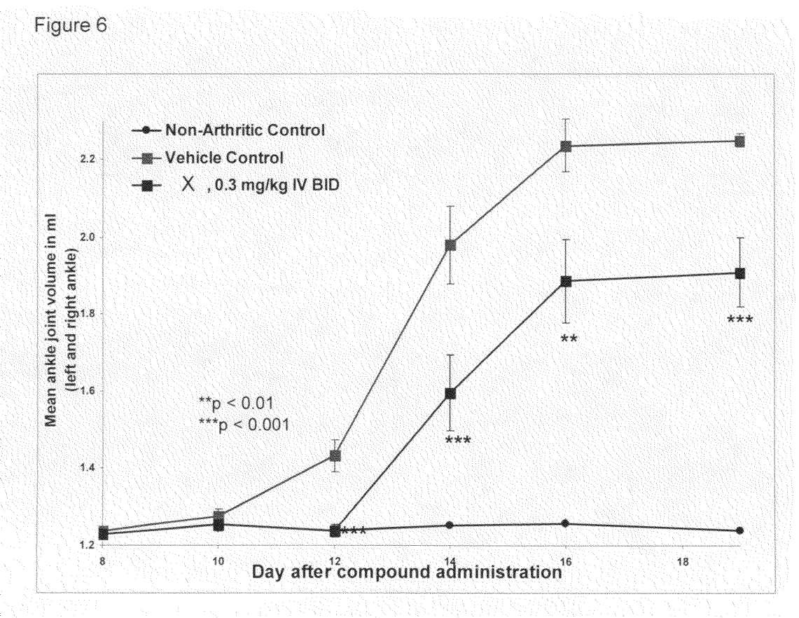

FIG. 6 shows that therapeutic dosing of Compound X inhibited arthritis in rats with CIA.

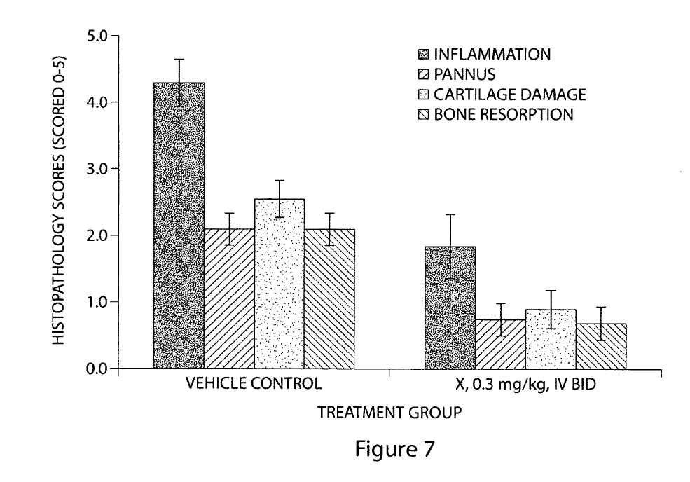

FIG. 7 shows that therapeutic dosing of Compound X significantly reduced knee histopathology scores in rats with CIA.

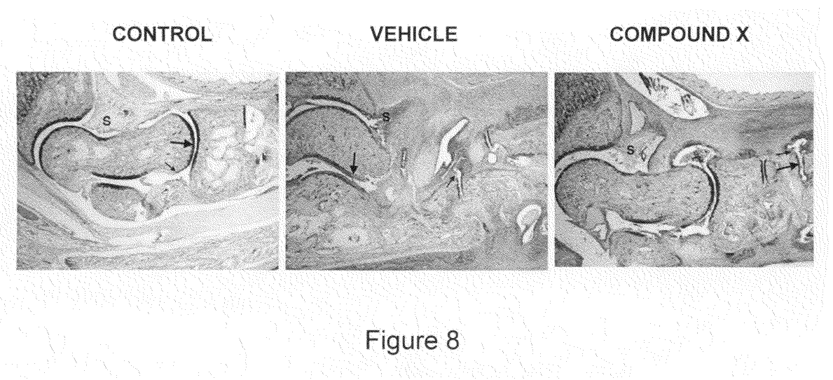

FIG. 8 shows that therapeutic dosing of Compound X protected bone resorption and joint damage in rats with CIA.

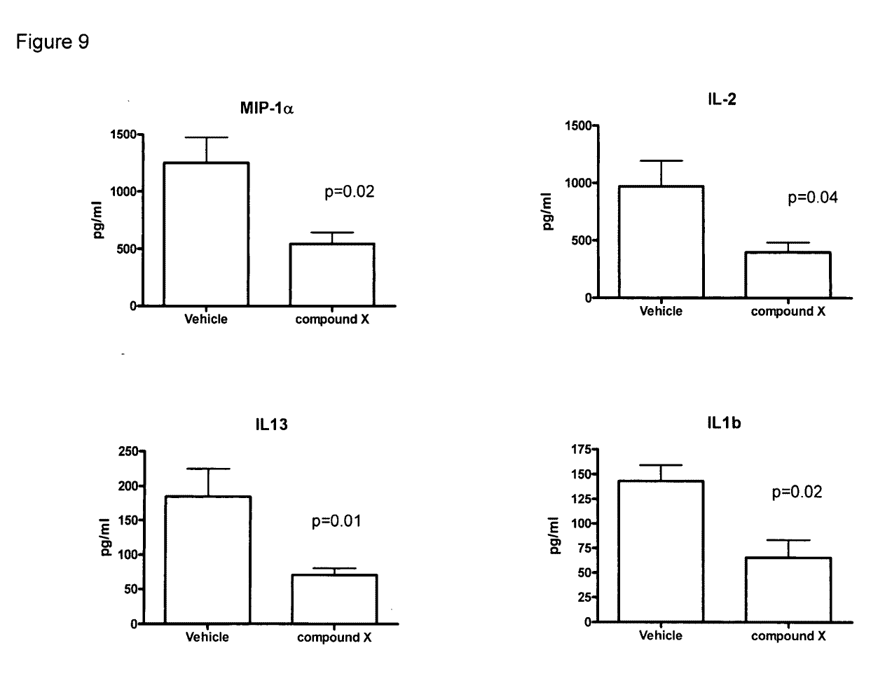

FIG. 9 shows that Compound X inhibited cytokine release of CD3-stimulated mouse splenocytes.

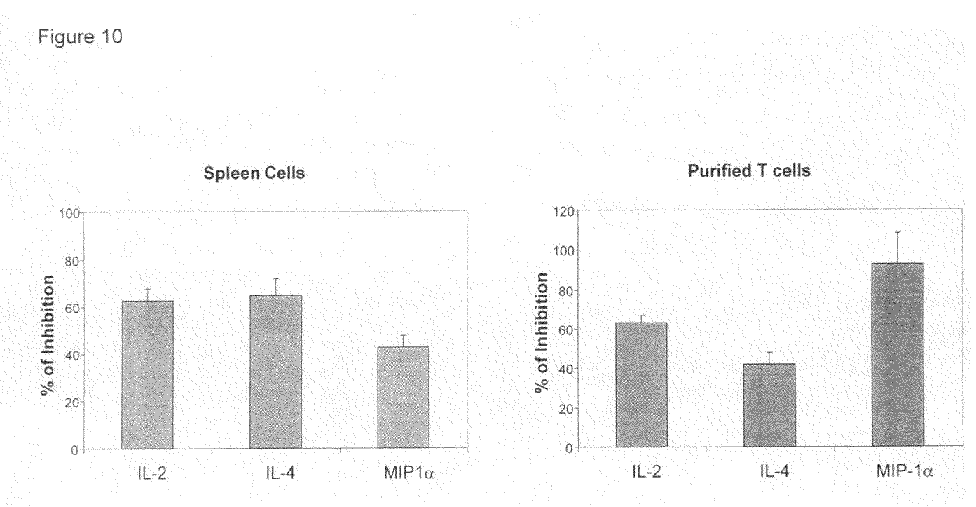

FIG. 10 shows that acute treatment of Compound X in vivo resulted in reduction of CD3-induced cytokine release.

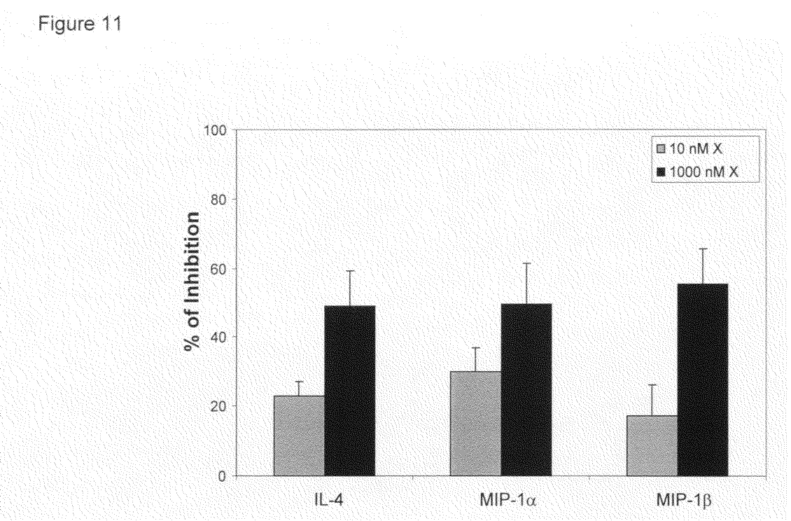

FIG. 11 shows that in vitro treatment with Compound X inhibited CD3-induced cytokine production of spleen cells.

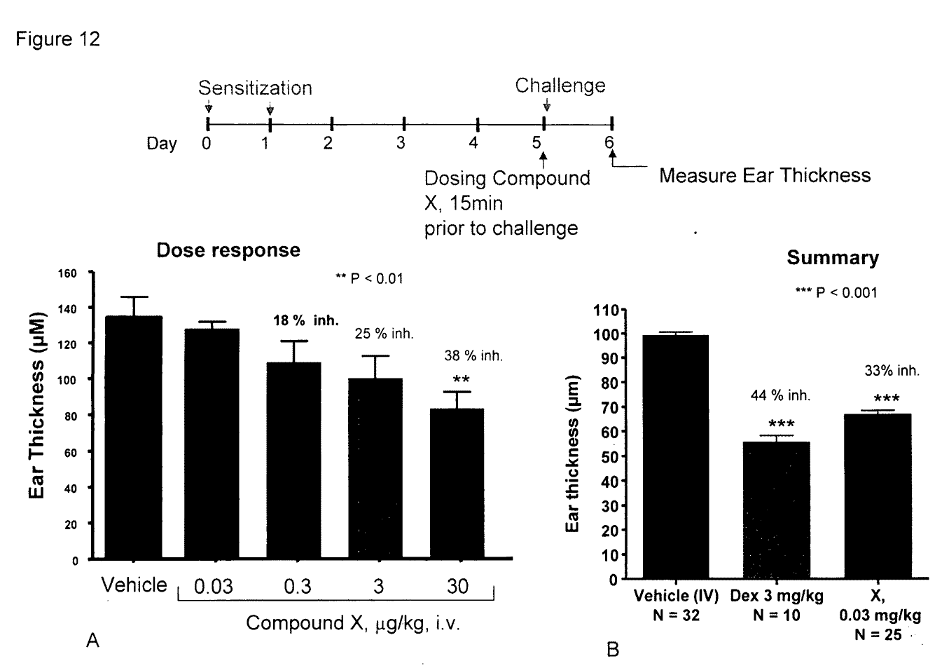

FIGS. 12a and 12b show that Compound X dose-dependently inhibited inflammation in murine DNFB-induced DTH model.

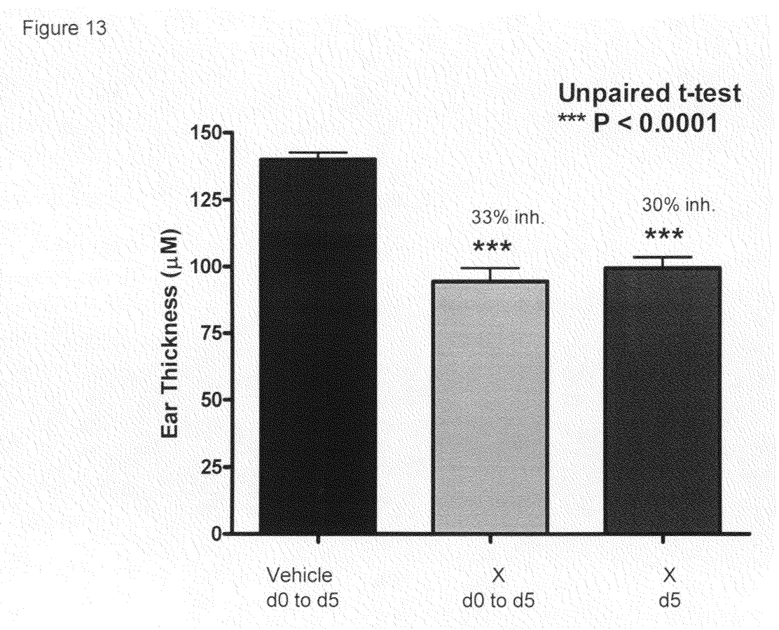

FIG. 13 shows that treatment with Compound X using two different regimens resulted in comparable and significant reduction of the DNFB-DTH response.

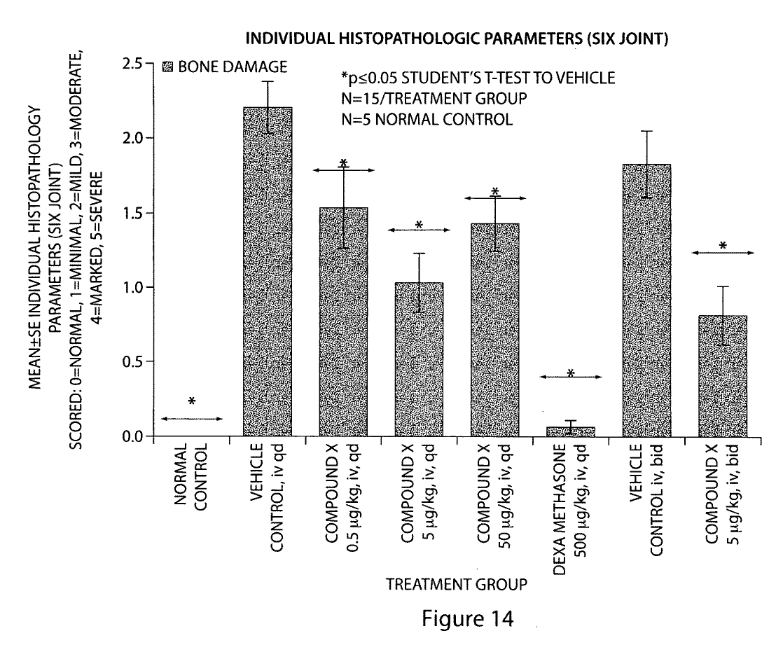

FIG. 14 shows the effects of Compound X on bone damage as was determined by histologic scoring in joints of mice with established Type II collagen arthritis.

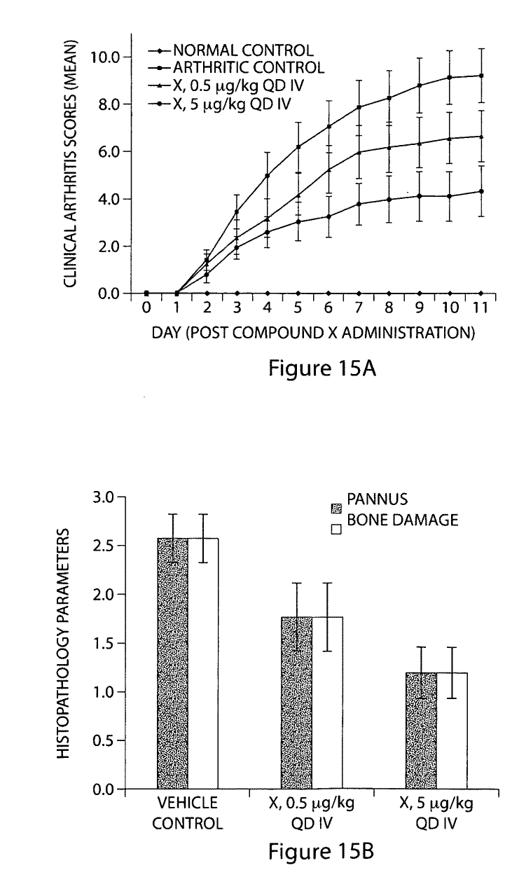

FIGS. 15a and 15b show the effects of Compound X on a) arthritis and b) pannus formation and bone loss in mice with established Type II collagen arthritis.

### DETAILED DESCRIPTION OF THE INVENTION

The present invention provides a method of inhibiting immune function in a patient, comprising administering to said patient a compound of formula A, a compound of any one of formulae 1-49, a lipoxin compound, an oxylipin compound, or a combination of aspirin and an omega-3 fatty acid.

The present invention provides a method of suppressing an immune response in a patient, comprising administering to said patient a compound of formula A, a compound of any one of formulae 1-49, a lipoxin compound, an oxylipin compound, or a combination of aspirin and an omega-3 fatty acid.

The present invention provides a method of treating an autoimmune disease or an autoimmune disorder in a patient, comprising administering to said patient a compound of formula A, a compound of any one of formulae 1-49, a lipoxin compound, an oxylipin compound, or a combination of aspirin and an omega-3 fatty acid.

In certain embodiments, the autoimmune disease or autoimmune disorder is of the type where the patient's own immune system damages one or more of the patient's tissues. In certain embodiments, the autoimmune disease or autoimmune disorder may be triggered by something within the patient or something within the patient's environment.

In certain embodiments, the autoimmune disease or autoimmune disorder of the present invention may be one which follows an initiating cause. For example, the autoimmune disease or autoimmune disorder may be one which is caused by an infection and/or some other initiating cause. Potential initiating causes may include old age, infection (such as parasitic infection), treatment with steroids, repeated vaccination with alum, pregnancy and/or cancers.

In certain embodiments, the autoimmune disease or autoimmune disorder may be organ-specific or non-organ-specific. Examples of such autoimmune diseases or autoimmune disorders include multiple sclerosis, arthritis (e.g., rheumatoid arthritis or juvenile arthritis), Crohn's disease, colitis ulcerosa, aplastic anemia, systemic lupus erythematosus (SLE or lupus), dermatomyositis, pernicious anemia, Addison's disease, ankylosing spondylitis, antiphospholipid syndrome, Churg-Strauss Syndrome, discoid lupus, fibromyalgia, Grave's Disease, myasthenia gravis, psoriasis, Reiter's Syndrome, rheumatic fever, sarcoidosis, scleroderma, Sjogren's Syndrome, stiff-man syndrome, thyroiditis, uveitis, vitiligo, Wegener's granulomatosis, graft rejection, insulin-dependent diabetes mellitus (e.g., Type I diabetes), and vascular disorders. In certain embodiments, the autoimmune disease or autoimmune disorder is selected from multiple sclerosis, aplastic anemia, systemic lupus erythematosus (SLE or lupus), dermatomyositis, pernicious anemia, Addison's disease, antiphospholipid syndrome, discoid lupus, fibromyalgia, Grave's Disease, myasthenia gravis, Reiter's Syndrome, sarcoidosis, scleroderma, Sjogren's Syndrome, stiff-man syndrome, vitiligo, graft rejection, insulin-dependent diabetes mellitus (e.g., Type I diabetes), or vascular disorders. In certain embodiments, the autoimmune disease or autoimmune disorder is selected from colitis ulcerosa, aplastic anemia, dermatomyositis, pernicious anemia, antiphospholipid syndrome, Churg-Strauss Syndrome, discoid lupus, fibromyalgia, Reiter's Syndrome, rheumatic fever, sarcoidosis, scleroderma, Sjogren's Syndrome, stiff-man syndrome, vitiligo, graft rejection, or vascular disorders. In certain embodiments, the autoimmune disease or autoimmune disorder is selected from multiple sclerosis, colitis ulcerosa, aplastic anemia, dermatomyositis, pernicious anemia, Addison's disease, antiphospholipid syndrome, Churg-Strauss Syndrome, discoid lupus, fibromyalgia, Grave's Disease, myasthenia gravis, psoriasis, Reiter's Syndrome, rheumatic fever, scleroderma, stiff-man syndrome, vitiligo, insulin-dependent diabetes mellitus (e.g., Type I diabetes), or vascular disorders. In certain embodiments, the autoimmune disease or autoimmune disorder is selected from aplastic anemia, dermatomyositis, pernicious anemia, antiphospholipid syndrome, discoid lupus, fibromyalgia, Reiter's Syndrome, sarcoidosis, scleroderma, stiff-man syndrome, vitiligo, or vascular disorders. In certain embodiments, the autoimmune disease or autoimmune disorder is selected from aplastic anemia, dermatomyositis, pernicious anemia, antiphospholipid syndrome, discoid lupus, fibromyalgia, Reiter's Syndrome, sarcoidosis, scleroderma, stiff-man syndrome, or vitiligo.

In certain embodiments wherein the autoimmune disease or autoimmune disorder is a vascular disorder, the vascular disorder may include any vascular disease or disorder which comprises an autoimmune element, for example one which is caused by an autoimmune response. Exemplary vascular disorders include one or more of Raynaud's disease and phenomenon, anterior uveitis, vasculitis, obliterative vascular disorder, atheroma formation (i.e., arteriosclerosis), arteritis (e.g., Takayasu arteritis, temporal arteritis/giant cell arteritis), myointimal hyperplasia (natural or following angioplasty), inflammatory and autoimmune thickening of the intima and/or muscular layer of blood vessels, inflammatory blood vessel lesions, atherosclerotic heart disease, reperfusion injury, cardiac conduction disturbances, myocarditis, and myocardial infarction. In certain embodiments, the vascular disorder is selected from one or more of Raynaud's disease and phenomenon, obliterative vascular disorder, arteritis (e.g., Takayasu arteritis, temporal arteritis/giant cell arteritis), myointimal hyperplasia (natural or following angioplasty), inflammatory and autoimmune thickening of the intima and/or muscular layer of blood vessels, inflammatory blood vessel lesions, and cardiac conduction disturbances. In certain embodiments, the vascular disorder is selected from one or more of Raynaud's disease and phenomenon, anterior uveitis, obliterative vascular disorder, arteritis (e.g., Takayasu arteritis, temporal arteritis/giant cell arteritis), myointimal hyperplasia (natural or following angioplasty), inflammatory and autoimmune thickening of the intima and/or muscular layer of blood vessels, inflammatory blood vessel lesions, atherosclerotic heart disease, cardiac conduction disturbances, and myocardial infarction. In certain embodiments, the vascular disorder is selected from one or more of Raynaud's disease and phenomenon, obliterative vascular disorder, arteritis (e.g., Takayasu arteritis, temporal arteritis/giant cell arteritis), myointimal hyperplasia (natural or following angioplasty), inflammatory and autoimmune thickening of the intima and/or muscular layer of blood vessels, inflammatory blood vessel lesions, atherosclerotic heart disease, cardiac conduction disturbances, and myocardial infarction. In certain embodiments, the vascular disorder is selected from one or more of Raynaud's disease and phenomenon, obliterative vascular disorder, arteritis (e.g., Takayasu arteritis, temporal arteritis/giant cell arteritis), myointimal hyperplasia (natural or following angioplasty), inflammatory and autoimmune thickening of the intima and/or muscular layer of blood vessels, inflammatory blood vessel lesions, and cardiac conduction disturbances.

In certain embodiments wherein the autoimmune disease or autoimmune disorder is graft rejection, the graft rejection may be chronic graft rejection. In certain embodiments of the present invention wherein a compound of formula A, a compound of any one of formulae 1-49, a lipoxin compound, an oxylipin compound, or a combination of aspirin and an omega-3 fatty acid is administered for the treatment of graft rejection, the administration of a compound of formula A, a compound of any one of formulae 1-49, a lipoxin compound, an oxylipin compound, or a combination of aspirin and an omega-3 fatty acid modulates immune responses to grafts (e.g., allografts or xenografts) where untreated rejection would otherwise lead to graft loss. Thus, a compound of formula A, a compound of any one of formulae 1-49, a lipoxin compound, an oxylipin compound, or a combination of aspirin and an omega-3 fatty acid may be used as a replacement for or in addition to the conventional immunosuppressant administered prior to, during and/or after transplantation. In certain embodiments, the graft rejection is in response to transplanting natural or artificial cells, islet cells, tissues (e.g., natural or artificial skin tissue), corneas, bone marrow, organs (e.g. kidney, liver, pancreas, lung, or heart), lenses, or pacemakers.

The present invention further provides a method of treating a disease, sequela or pathological condition mediated by an activation of the immune system in a patient, comprising administering to said patient a compound of formula A, a compound of any one of formulae 1-49, a lipoxin compound, an oxylipin compound, or a combination of aspirin and an omega-3 fatty acid. In certain embodiments, diseases, sequelae and pathological conditions mediated by an activation of the immune system include capillary leakage, pulmonary failure, sepsis, endotoxic shock, or sequelae of tissue damage. In certain embodiments, diseases, sequelae and pathological conditions mediated by an activation of the immune system are selected from capillary leakage or sepsis.

Compounds suitable for use in methods of the invention include those of Formula A,

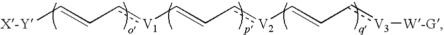

wherein:

each of W′ and Y′ is a bond or a linker independently selected from a ring containing up to 20 atoms or a chain of up to 20 atoms, provided that W′ and Y′ can independently include one or more nitrogen, oxygen, sulfur or phosphorous atoms, further provided that W′ and Y′ can independently include one or more substituents independently selected from hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, or sulfonyl, further provided that W′ and Y′ can independently contain one or more fused carbocyclic, heterocyclic, aryl or heteroaryl rings, and further provided that when o′ is 0, and V₁ is

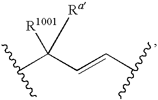

Y′ is connected to V₁ via a carbon atom;

V₁ is selected from

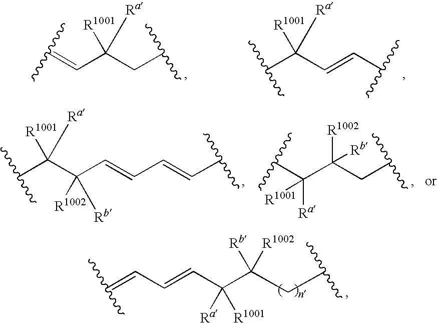

wherein when q′ is 0 and V₃ is a bond, n′ is 0 or 1; otherwise n′ is 1;

V₂ is selected from a bond,

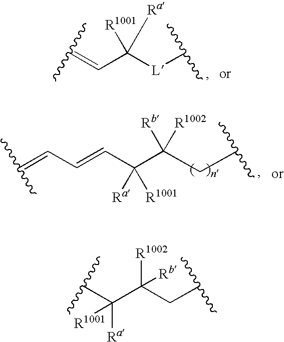

wherein:
L′ is selected from —C(R¹⁰⁰³)(R¹⁰⁰⁴)—, wherein each of R¹⁰⁰³ and R¹⁰⁰⁴ is independently selected from hydrogen, alkyl, alkenyl, alkynyl, perfluoroalkyl, alkoxy, aryl or heteroaryl, or R¹⁰⁰³ and R¹⁰⁰⁴ are connected together to form a carbocyclic or heterocyclic ring; when V₃ is

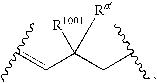

L′ is additionally selected from W′; and n′ is 0 or 1;

V₃ is selected from a bond or

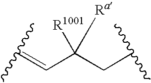

wherein:
each R¹⁰⁰¹ and R¹⁰⁰² is independently for each occurrence selected from hydrogen, alkyl, alkenyl, alkynyl, aryl, heteroaryl, alkylaryl, alkoxy, or halo, wherein said alkyl- or aryl-containing moiety is optionally substituted with up to 3 independently selected substituents;
each of R^{a}′ and R^{b}′ is independently for each occurrence selected from —OR′ or —N(R′)₂, or adjacent R^{a}′ and R^{b}′ are taken together to form an epoxide ring having a cis or trans configuration, wherein each R′ is independently selected from hydrogen, alkyl, alkenyl, alkynyl, aryl, heteroaryl, acyl, silyl, alkoxyacyl, aminoacyl, aminocarbonyl, alkoxycarbonyl, or a protecting group;

or when V₁ is

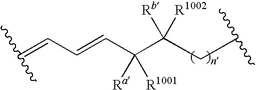

### and V₂ is

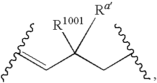

R¹⁰⁰² and R^{b}′ are both hydrogen;

X′ is selected from —CN, —C(NH)N(R″)(R″), —C(S)-A′, —C(S)R″, —C(O)-A′, —C(O)—R″, —C(O)—SR″, —C(O)—NH—S(O)₂—R″, —S(O)₂-A′, —S(O)₂—R″, S(O)₂N(R″)(R″), —P(O)₂-A′, —PO(OR″)-A′, -tetrazole, alkyltetrazole, or —CH₂OH, wherein
A′ is selected from —OR″, —N(R″)(R″) or —OM′;
each R″ is independently selected from hydrogen, alkyl, aryl, arylalkyl, heteroaryl, heteroarylalkyl or a detectable label molecule, wherein any alkyl-, aryl- or heteroaryl-containing moiety is optionally substituted with up to 3 independently selected substituents; and
M′ is a cation;

G′ is selected from hydrogen, halo, hydroxy, alkyl, aryl, arylalkyl, heteroaryl, heteroarylalkyl, alkoxy, aryloxy, carboxy, amino, alkylamino, dialkylamino, acylamino, carboxamido or a detectable label molecule, wherein any alkyl-, aryl- or heteroaryl-containing moiety is optionally substituted with up to 3 independently selected substituents;

o′ is 0, 1, 2, 3, 4, or 5;

p′ is 0, 1, 2, 3, 4, or 5;

q′ is 0, 1, or 2; and

o′+p′+q′ is 1, 2, 3, 4, 5 or 6;

wherein:

if V₂ is a bond, then q′ is 0, and V₃ is a bond;

if V₃ is

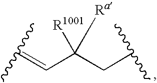

then o′ is 0, V₁ is

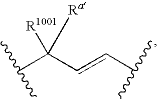

p′ is 1 and V₂ is

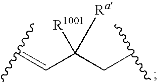

any acyclic double bond may be in a cis or a trans configuration or is optionally replaced by a triple bond; and

either one

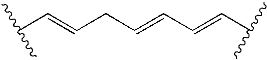

portion of the compound, if present, is optionally replaced by

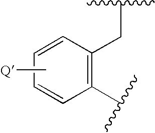

or one

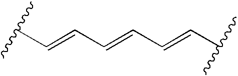

portion of the compound, if present, is optionally replaced by

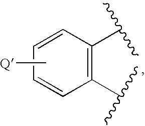

wherein Q′ represents one or more substituents and each Q′ is independently selected from halo, alkyl, alkenyl, alkynyl, cycloalkyl, aryl, heteroaryl, alkoxy, aryloxy, alkylcarbonyl, arylcarbonyl, alkoxycarbonyl, aryloxycarbonyl, amino, hydroxy, cyano, carboxyl, alkoxycarbonyloxy, aryloxycarbonyloxy or aminocarbonyl.

In certain embodiments, V₁ is selected from

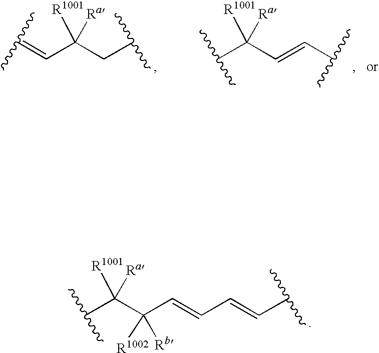

In certain embodiments, V₂ is selected from a bond,

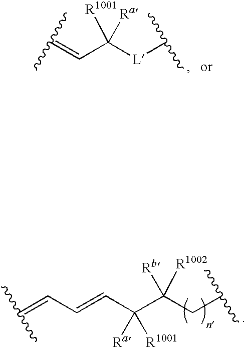

In certain embodiments, when q′ is 0 and V₃ is a bond, n′ is 0 or 1; otherwise n′ is 1.

In certain embodiments, p′ is 0, 1, 2, 3, or 5.

In certain embodiments, q′ is 0 or 1.

In certain embodiments, if V₁ is

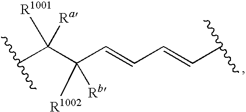

then o′ is 0 or 1, p′ is 1 or 2, o′+p′ is 1 or 2, V₂ is

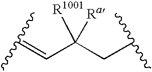

and V₃ is a bond.

In certain embodiments, if V₁ is

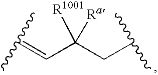

then o′ is 3, 4 or 5, p′ is 0, 1 or 2, o′+p′ is 4 or 5, and V₂ is a bond.

In certain embodiments, if V₂ is a bond, then o′ is 0, 3, 4 or 5; p′ is 0, 1, 2 or 5, o′+p′ is 4 or 5, q′ is 0, and V₃ is a bond.

In certain embodiments, each of W′ and Y′ is independently selected from a bond or lower alkyl or heteroalkyl optionally substituted with one or more substituents independently selected from alkenyl, alkynyl, aryl, chloro, iodo, bromo, fluoro, hydroxy, amino, or oxo.

Compounds suitable for use in methods of the invention include those of Formula 1,

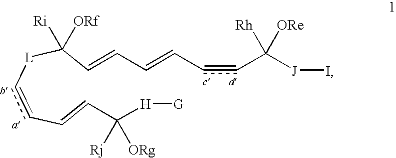

wherein
Carbons a′ and b′ are connected by a double bond or a triple bond;
Carbons c′ and d′ are connected by a double bond or a triple bond;
Re, Rf, and Rg are independently selected from hydrogen, alkyl, alkenyl, alkynyl, aryl, heteroaryl, acyl (e.g., alkoxyacyl, aminoacyl), aminocarbonyl, alkoxycarbonyl, or silyl;
Rh, Ri and Rj are independently selected from hydrogen, alkyl, alkenyl, alkynyl, perfluoroalkyl, aryl or heteroaryl;
I is selected from —C(O)-E, —SO₂-E, —PO(OR)-E, where E is hydroxy, alkoxy, aryloxy, amino, alkylamino, dialkylamino, or arylamino; and R is hydrogen or alkyl;
J, L and H are linkers independently selected from a ring containing up to 20 atoms or a chain of up to 20 atoms, provided that J, L and H can independently include one or more nitrogen, oxygen, sulfur or phosphorous atoms, and further provided that J, L and H can independently include one or more substituents selected from hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, and sulfonyl, and further provided that J, L and H can also contain one or more fused carbocyclic, heterocyclic, aryl or heteroaryl rings, and provided that linker J is connected to the adjacent C(R)OR group via a carbon atom;
G is selected from hydrogen, alkyl, perfluoroalkyl, alkenyl, alkynyl, aryl, heteroaryl, chloro, iodo, bromo, fluoro, hydroxy, alkoxy, aryloxy, carboxy, amino, alkylamino, dialkylamino, acylamino, or carboxamido; or pharmaceutically acceptable salts thereof.

In certain embodiments, a pharmaceutically acceptable salt of the compound is formed by derivatizing E, wherein E is —OM, where M is a cation selected from ammonium, tetra-alkyl ammonium, Na, K, Mg, and Zn.

In certain embodiments, a compound of formula 1 is represented by formula 2,

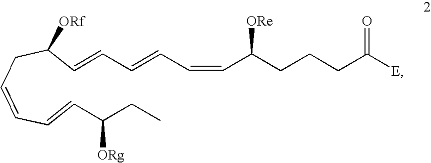

wherein

E, Re, Rf, and Rg are as defined above.
In certain embodiments, a pharmaceutically acceptable salt of the compound is formed by derivatizing E, wherein E is —OM, where M is a cation selected from ammonium, tetra-alkyl ammonium, Na, K, Mg, and Zn.

Exemplary compounds of formula 2 include:

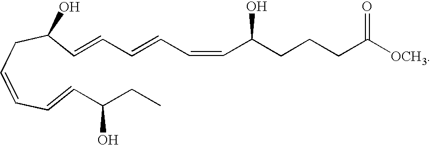

In certain embodiments, a compound of formula 1 is represented by formula 3,

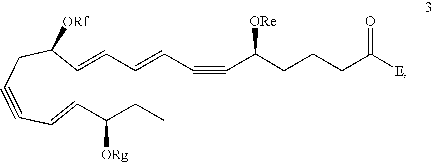

wherein

E, Re, Rf, and Rg are as defined above.
In certain embodiments, a pharmaceutically acceptable salt of the compound is formed by derivatizing E, wherein E is —OM, where M is a cation selected from ammonium, tetra-alkyl ammonium, Na, K, Mg, and Zn.
Exemplary compounds of formula 3 include:

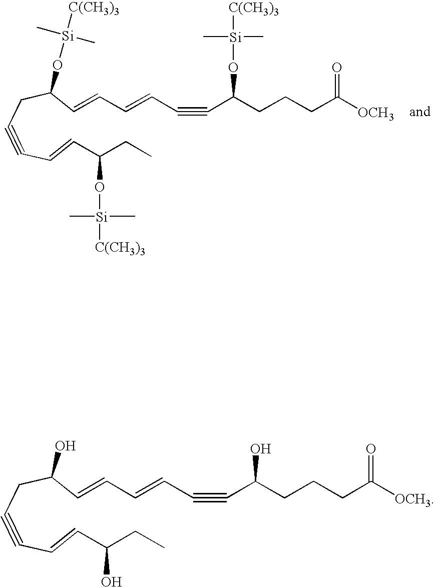

Further exemplary compounds of formula 1 include Compound X,

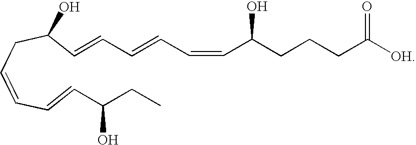

Other compounds suitable for use in methods of the invention include those of Formula 4,

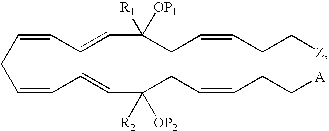

wherein

A is H or —OP₄;

P₁, P₂ and P₄ each individually is a protecting group or hydrogen atom;

R₁ and R₂ each individually is a substituted or unsubstituted, branched or unbranched alkyl, alkenyl, or alkynyl group, substituted or unsubstituted aryl group, substituted or unsubstituted, branched or unbranched alkylaryl group, halogen atom, hydrogen atom;

Z is —C(O)OR^{d}, —C(O)NR^{c}R^{c}, —C(O)H, —C(NH)NR^{c}R^{c}, —C(S)H, —C(S)OR^{d}, —C(S)NR^{c}R^{c}, —CN, preferably a carboxylic acid, ester, amide, thioester, thiocarboxamide or a nitrile;

each R^{a}, if present, is independently selected from hydrogen, (C1-C6) alkyl, (C2-C6) alkenyl, (C2-C6) alkynyl, (C3-C8) cycloalkyl, cyclohexyl, (C4-C11) cycloalkylalkyl, (C5-C10) aryl, phenyl, (C6-C16) arylalkyl, benzyl, 2-6 membered heteroalkyl, 3-8 membered heterocyclyl, morpholinyl, piperazinyl, homopiperazinyl, piperidinyl, 4-11 membered heterocyclylalkyl, 5-10 membered heteroaryl and 6-16 membered heteroarylalkyl;

each R^{b}, if present, is a suitable group independently selected from ═O, —OR^{d}, (C1-C3) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, —[NR^{a}C(O)]ₙR^{d}, —[NHC(O)]ₙOR^{d}, —[NR^{a}C(O)]ₙOR^{d}, [NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c};

each R^{c}, if present, is independently a protecting group or R^{a}, or, alternatively, two R^{c} taken together with the nitrogen atom to they are bonded form a 5 to 8-membered heterocyclyl or heteroaryl which optionally including one or more additional heteroatoms and optionally substituted with one or more of the same or different R^{a} or suitable R^{b} groups;

each n independently is an integer from 0 to 3;

each R^{d} independently is a protecting group or R^{a}; or pharmaceutically acceptable salts thereof.

Other compounds suitable for use in methods of the invention include those of Formula 5,

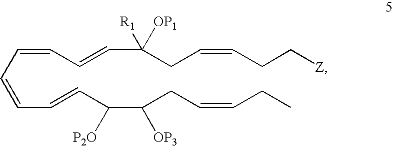

or pharmaceutically acceptable salts thereof, wherein
P₃ is a protecting group or hydrogen atom; and

P₁, P₂, R₁ and Z are as defined above in formula 4.

Exemplary compounds of formula 5 include compound 5a,

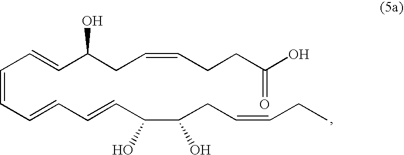

and pharmaceutically acceptable salts and esters thereof.

Other compounds suitable for use in methods of the invention include those of Formula 6,

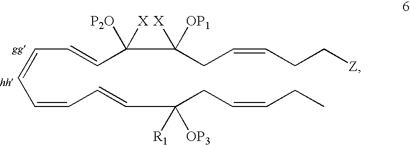

or pharmaceutically acceptable salts thereof, wherein
the stereochemistry of the carbon gg′ to carbon hh′ bond is cis or trans;
each X represents hydrogen or taken together both X groups represent one substituted or unsubstituted methylene, an oxygen atom, a substituted or unsubstituted N atom, or a sulfur atom such that a three-membered ring is formed; and
P₁, P₂, P₃, R₁ and Z are as defined above.

Exemplary compounds of formula 6 include compound 6a,

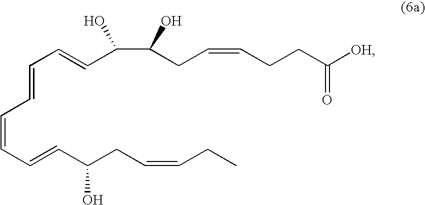

and pharmaceutically acceptable salts and esters thereof.

Other compounds suitable for use in methods of the invention include those of Formula 7,

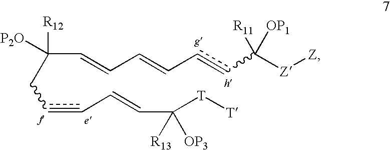

or pharmaceutically acceptable salts thereof, wherein
Carbons e′ and f′ are connected by a double bond or a triple bond, and when carbon e′ is connected to carbon f′ through a double bond the stereochemistry is cis or trans;
Carbons g′ and h′ are connected by a double bond or a triple bond and when carbon g′ is connected to carbon h′ through a double bond the stereochemistry is cis or trans;
m is 0 or 1;
T′ is hydrogen, (C1-C6) alkyl, (C2-C6) alkenyl, (C2-C6) alkynyl, (C5-C14) aryl, (C6-C16) arylalkyl, 5-14 membered heteroaryl, 6-16 membered heteroarylalkyl, or —CH═CHCH₂CH₃;
T is —(CH₂)_{q}— or —(CH₂)_{q}—O—, where q is an integer from 0 to 6;
Z′ is (C1-C6) alkylene optionally substituted with 1, 2, 3, 4, 5 or 6 of the same or different halogen atoms, —(CH₂)ₚ—O—CH₂— or —(CH₂)ₘ—S—CH₂—, where p is an integer from 0 to 4;
R₁₁, R₁₂ and R₁₃ each individually is substituted or unsubstituted, branched or unbranched alkyl, alkenyl, or alkynyl group, substituted or unsubstituted aryl group, substituted or unsubstituted, branched or unbranched alkylaryl group, C₁₋₄alkoxy, halogen atom, —CH₂R₁₄, —CHR₁₄R₁₄, —CR₁₄R₁₄R₁₄ or a hydrogen atom;
R₁₄ is independently for each occurrence selected from —CN, —NO₂ or halogen;
P₁, P₂, P₃, and Z are as defined above.

Other compounds suitable for use in methods of the invention include those of Formula 8,

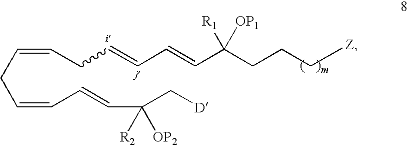

or pharmaceutically acceptable salts thereof, wherein
the stereochemistry of the carbon i′ to carbon j′ bond is cis or trans;
m is 0 or 1;
D′ is CH₃, —CH═CHCH₂U or —CH═CHCH₂CH₂A;
U is a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, alkoxycarbonyloxy, and aryloxycarbonyloxy group;
A is H or —OP₄;
P₁, P₂, P₄, R₁, R₂ and Z are as defined above.

Other compounds suitable for use in methods of the invention include those of Formula 9,

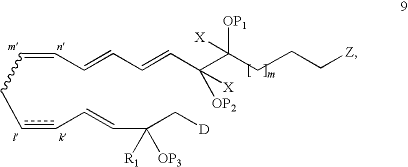

or pharmaceutically acceptable salts thereof, wherein
Carbons k′ and l′ are connected by a double bond or a triple bond;
the stereochemistry of the carbon m′ to carbon n′ double bond is cis or trans;
m is 0 or 1;

### D is —CH₃ or —CH═CHCH₂CH₃;

P₁, P₂, P₃, R₁, X, and Z are as defined above.

Exemplary compounds of formula 9 include compound 9a,

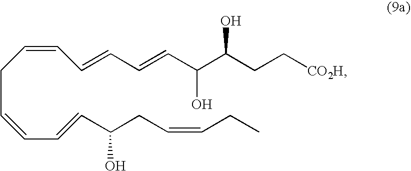

and pharmaceutically acceptable salts and esters thereof.

Other compounds suitable for use in methods of the invention include those of Formula 10,

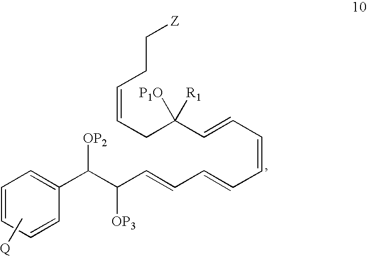

or pharmaceutically acceptable salts thereof, wherein
P₁, P₂, P₃, R₁ and Z are as defined above; and
Q represents one or more substituents and each Q individually, if present, is a halogen atom or a branched or unbranched, substituted or unsubstituted alkyl, alkenyl, alkynyl, cycloalkyl, aryl, alkoxy, aryloxy, alkylcarbonyl, arylcarbonyl, alkoxycarbonyl, aryloxycarbonyl, amino, hydroxy, cyano, carboxyl, alkoxycarbonyloxy, aryloxycarbonyloxy or aminocarbonyl group.

Other compounds suitable for use in methods of the invention include those of Formula 11,

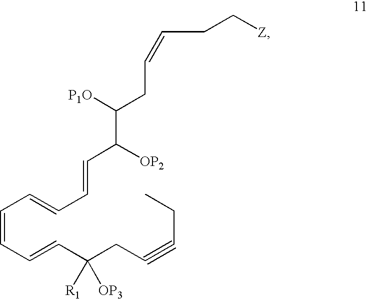

or pharmaceutically acceptable salts thereof, wherein
P₁, P₂, P₃, R₁, and Z are as defined above.

Other compounds suitable for use in methods of the invention include those of Formula 12,

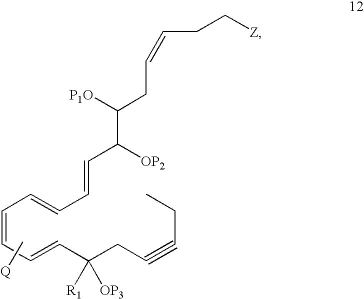

or pharmaceutically acceptable salts thereof, wherein
P₁, P₂, P₃, Q, R₁, and Z are as defined above.

Other compounds suitable for use in methods of the invention include those of Formula 13,

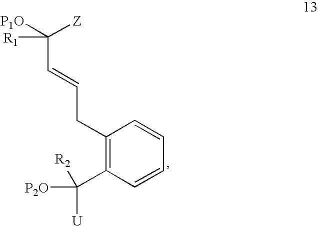

or pharmaceutically acceptable salts thereof, wherein
P₁, P₂, R₁, R₂, U, and Z are as defined above.

Other compounds suitable for use in methods of the invention include those of Formula 14,

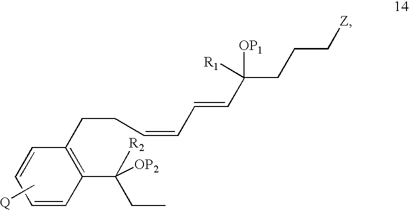

or pharmaceutically acceptable salts thereof, wherein
P₁, P₂, R₁, R₂, Q, and Z are as defined above.

Other compounds suitable for use in methods of the invention include those of Formula 15,

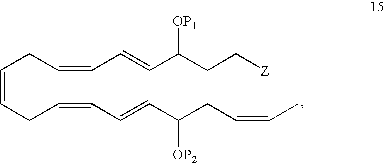

or pharmaceutically acceptable salts thereof, wherein
P₁, P₂, and Z are as defined above.

Other compounds suitable for use in methods of the invention include those of Formula 16,

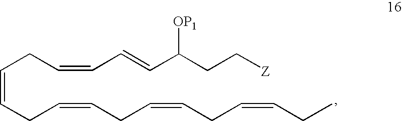

or pharmaceutically acceptable salts thereof, wherein
P₁ and Z are as defined above.

Other compounds suitable for use in methods of the invention include those of Formula 17,

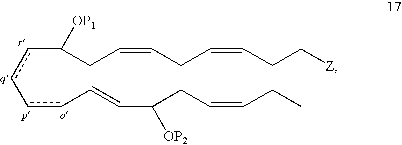

or pharmaceutically acceptable salts thereof, wherein
Carbons o′ and p′ are connected by a single or a double bond;
Carbons q′ and r′ are connected by a single or a double bond; and
P₁, P₂, and Z are as defined above.

Other compounds suitable for use in methods of the invention include those of Formula 18,

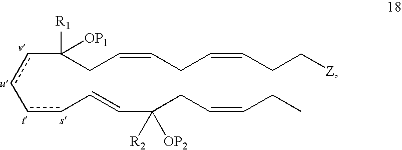

or pharmaceutically acceptable salts thereof, wherein
the stereochemistry of the carbon s′ to carbon t′ double bond is cis or trans;
the stereochemistry of the carbon u′ to carbon v′ double bond is cis or trans; and
P₁, P₂, R₁, R₂, and Z are as defined above.

Other compounds suitable for use in methods of the invention include those of Formula 19,

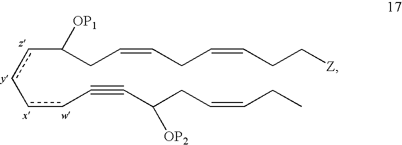

or pharmaceutically acceptable salts thereof, wherein
Carbons w′ and x′ are connected by a single or a double bond;
Carbons y′ and z′ are connected by a single or a double bond; and
P₁, P₂, and Z are as defined above.

In certain embodiments of formulae 4 to 19, each R^{b}, if present, is a suitable group independently selected from ═O, —OR^{d}, (C1-C3) haloalkyloxy, —OCF₃, ═S, —SR^{d}, ═NR^{d}, ═NOR^{d}, —NR^{c}R^{c}, halogen, —CF₃, —CN, —NC, —OCN, —SCN, —NO, —NO₂, ═N₂, —N₃, —S(O)R^{d}, —S(O)₂R^{d}, —S(O)₂OR^{d}, —S(O)NR^{c}R^{c}, —S(O)₂NR^{c}R^{c}, —OS(O)R^{d}, —OS(O)₂R^{d}, —OS(O)₂OR^{d}, —OS(O)₂NR^{c}R^{c}, —C(O)R^{d}, —C(O)OR^{d}, —C(O)NR^{c}R^{c}, —C(NH)NR^{c}R^{c}, —C(NR^{a})NR^{c}R^{c}, —C(NOH)R^{a}, —C(NOH)NR^{c}R^{c}, —OC(O)R^{d}, —OC(O)OR^{d}, —OC(O)NR^{c}R^{c}, —OC(NH)NR^{c}R^{c}, —OC(NR^{a})NR^{c}R^{c}, —[NHC(O)]ₙR^{d}, [NR^{a}C(O)]ₙR^{d}, —[NHC(O)]ₙOR^{d}, [NHC(O)]ₙNR^{c}R^{c}, —[NR^{a}C(O)]ₙNR^{c}R^{c}, —[NHC(NH)]ₙNR^{c}R^{c} and —[NR^{a}C(NR^{a})]ₙNR^{c}R^{c}.

Other compounds suitable for use in methods of the invention include those of Formula 20,

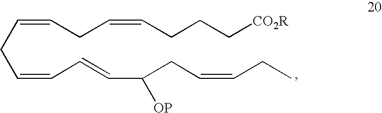

Formula 21,

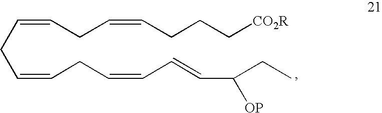

Formula 22,

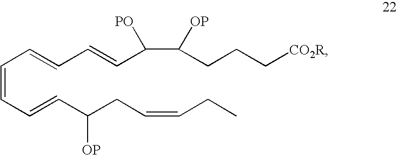

Formula 23,

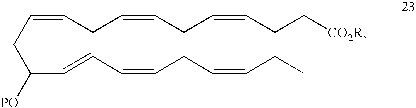

Formula 24,

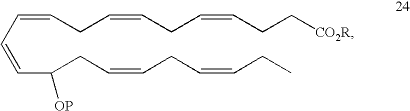

Formula 25,

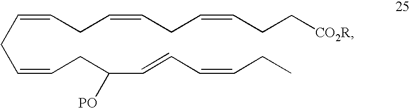

Formula 26,

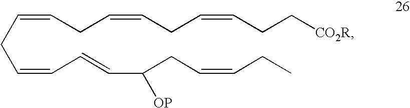

Formula 27,

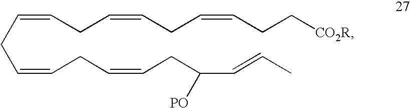

Formula 28,

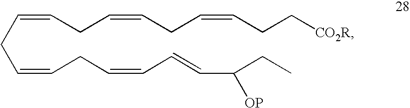

or pharmaceutically acceptable salts of any of the above, wherein
each P is individually selected from H or a protecting group; and
R is H, C₁₋₆alkyl (e.g., methyl, ethyl, glycerol), C₂₋₆alkenyl or C₂₋₆alkynyl.

Other compounds suitable for use in methods of the invention include those of Formula 29,

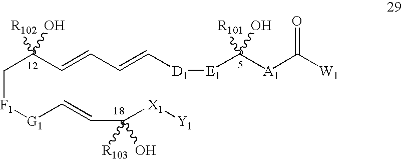

and pharmaceutically acceptable salts, hydrates and solvates thereof, wherein:
D₁-E₁ and F₁-G₁ are independently are cis or trans —C═C— or —C≡C—;
R₁₀₁, R₁₀₂ and R₁₀₃ are independently selected from hydrogen, (C1-C4) straight-chained or branched alkyl, (C2-C4) alkenyl, (C2-C4) alkynyl, (C1-C4) alkoxy, —CH₂R₁₀₄, —CHR₁₀₄R₁₀₄ and —CR₁₀₄R₁₀₄R₁₀₄;
each R₁₀₄ is independently selected from CN, —NO₂ and halogen;
W₁ is selected from —R₁₀₅, —OR₁₀₅, —SR₁₀₅ and —NR₁₀₅R₁₀₅;
each R₁₀₅ is independently selected from hydrogen, (C1-C6)alkyl, (C2-C6)alkenyl or (C2-C6) alkynyl optionally substituted with one or more of the same or different R groups, (C5-C14) aryl optionally substituted with one or more of the same or different R groups, phenyl optionally substituted with one or more of the same or different R groups, (C6-C16) arylalkyl optionally substituted with one or more of the same or different R groups, 5-14 membered heteroaryl optionally substituted with one or more of the same or different R groups, 6-16 membered heteroarylalkyl optionally substituted with one or more of the same or different R groups and a detectable label molecule;
A₁ is selected from (C1-C6) alkylene optionally substituted with 1, 2, 3, 4, 5 or 6 of the same or different halogen atoms, —(CH₂)ₘ—O—CH₂— and —(CH₂)ₘ—S—CH₂—, where m is an integer from 0 to 4;
X₁ is selected from —(CH₂)ₙ— and —(CH₂)ₙ—O—, where n is an integer from 0 to 6;
Y₁ is selected from hydrogen, (C1-C6)alkyl, (C2-C6)alkenyl, or (C2-C6)alkynyl, optionally substituted with one or more of the same or different R₁₀₀ groups, (C5-C14) aryl optionally substituted with one or more of the same or different R₁₀₀ groups, phenyl, optionally substituted with one or more of the same or different R₁₀₀ groups, (C6-C16)arylalkyl optionally substituted with one or more of the same or different R₁₀₀ groups, 5-14 membered heteroaryl optionally substituted with one or more of the same or different R₁₀₀ groups, 6-16 membered heteroarylalkyl optionally substituted with one or more of the same or different R₁₀₀ groups and a detectable label molecule;
each R₁₀₀ is independently selected from an electronegative group, ═O, —OR^{a1}, (C1-C3) haloalkyloxy, ═S, —SR^{a1}, ═NR^{a1}, ═NONR^{a1}, —NR^{c1}R^{c1}, halogen, —CF₃, —CN, —NC, —OCN, —SCN, —NO, —NO₂, ═N₂, —N₃, —S(O)R^{a1}, —S(O)₂R^{a1}, —S(O)₂OR^{a1}, —S(O)₂NR^{c1}R^{c1}, —OS(O)R^{a1}, —OS(O)₂R^{a1}, —OS(O)₂OR^{a1}, —OS(O)₂NR^{c1}R^{c1}, —C(O)R^{a1}, —C(O)OR^{a1}, —C(O)NR^{c1}R^{c1}, —C(NH)NR^{c1}R^{c1}, —OC(O)R^{a1}, OC(O)OR^{a1}, —OC(O)NR^{c1}R^{c1}, —OC(NH)NR^{c1}R^{c1}, —NHC(O)R^{a1}, —NHC(O)OR^{a1}, —NHC(O)NR^{c1}R^{c1} and —NHC(NH)NR^{c1}R^{c1};
each R^{a1} is independently selected from hydrogen, (C1-C4) alkyl, (C2-C4) alkenyl or (C2-C4) alkynyl; and
each R^{c1} is independently an R^{a1} or, alternatively, R^{c1}R^{c1} taken together with the nitrogen atom to which it is bonded forms a 5 or 6 membered ring.

In certain embodiments of Formula 29, when X₁-Y₁ is —CH₂CH₃, then at least one of R₁₀₁, R₁₀₂ or R₁₀₃ is other than hydrogen.

In certain embodiments, a compound of Formula 29 is represented by Formula 30,

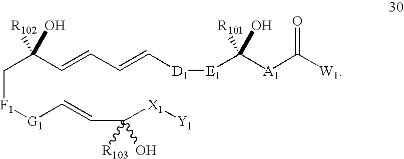

Other compounds suitable for use in methods of the invention include those of Formulae 31 to 37

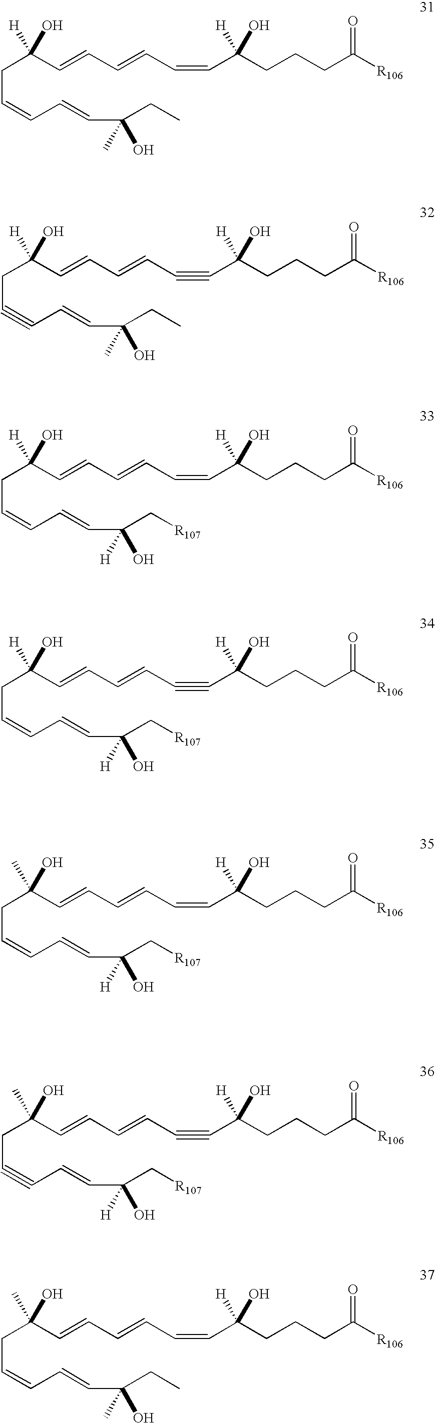

and pharmaceutically acceptable salts, hydrates and solvates thereof,

wherein

### R₁₀₆ is —OH, —OCH₃, —OCH(CH₃)₂ or —NHCH₂CH₃; and

### R₁₀₇ is

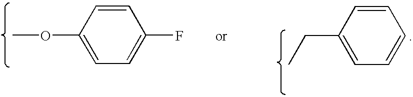

Other compounds suitable for use in methods of the invention include those of Formula 38,

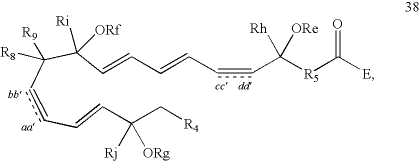

wherein
Carbons aa′ and bb′ are connected by a double bond or a triple bond;
Carbons cc′ and dd′ are connected by a double bond or a triple bond;
Re, Rf, and Rg are independently selected from hydrogen, alkyl, alkenyl, alkynyl, aryl, heteroaryl, acyl (e.g., alkoxyacyl, aminoacyl), aminocarbonyl, alkoxycarbonyl, or silyl;
E is hydroxyl, alkoxy, aryloxy, amino, alkylamino, dialkylamino, or arylamino;
Rh, Ri and Rj are independently selected from hydrogen, alkyl, alkenyl, alkynyl, perfluoroalkyl, aryl or heteroaryl;
R₄ is selected from hydrogen, alkyl, perfluoroalkyl, alkenyl, alkynyl, aryl, heteroaryl, fluoro, hydroxyl, alkoxy, aryloxy;
R₅ is selected from i-iv as follows: i) CH₂CH(R₆)CH₂, where R₆ is hydrogen, alkyl, alkenyl, alkynyl, perfluoroalkyl, aryl, heteroaryl, fluoro, hydroxyl or alkoxy; ii) CH₂C(R₆R₇)CH₂, where R₆ and R₇ are each independently alkyl, alkenyl, alkynyl, perfluoroalkyl, aryl, or fluoro, or R₆ and R₇ are connected together to form a carbocyclic or heterocyclic ring; iii) CH₂OCH₂, CH₂C(O)CH₂, or CH₂CH₂; or iv) R₅ is a carbocyclic, heterocyclic, aryl or heteroaryl ring; and
R₈ and R₉ are independently selected from hydrogen, alkyl, alkenyl, alkynyl, perfluoroalkyl, alkoxy, aryl or heteroaryl, or R₈ and R₉ are connected together to form a carbocyclic or heterocyclic ring;
or pharmaceutically acceptable salts thereof.

In certain embodiments R₈ and R₉ are hydrogen.

In certain embodiments, a pharmaceutically acceptable salt of the compound is formed by derivatizing E, wherein E is —OM, where M is a cation selected from ammonium, tetra-alkyl ammonium, Na, K, Mg, and Zn.

Other compounds suitable for use in methods of the invention include those of Formulae 39-44,

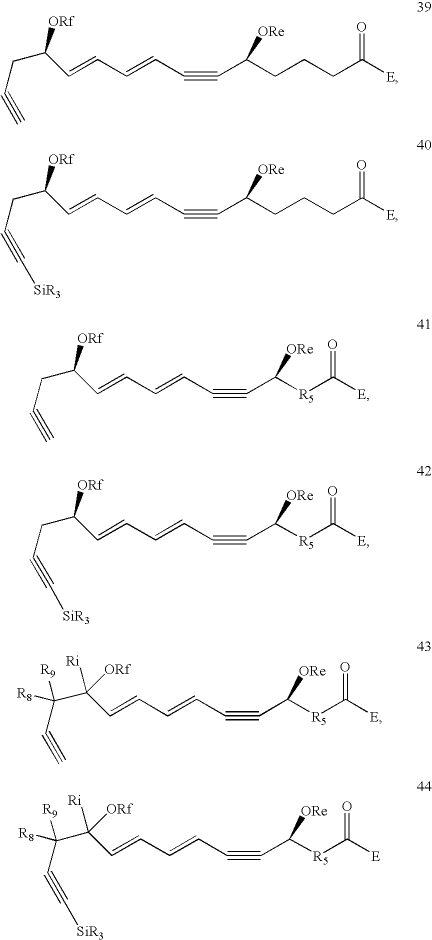

and pharmaceutically acceptable salts thereof, wherein
Re, Rf, E, Ri, R₅, R₈ and R₉ are as defined above.

Exemplary compounds of formulae 39, 41, and 43 include:

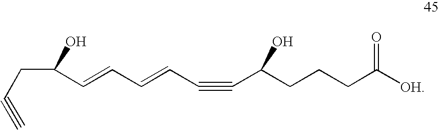

In certain embodiments, a pharmaceutically acceptable salt of the compound is formed by derivatizing E, wherein E is —OM, where M is a cation selected from ammonium, tetra-alkyl ammonium, Na, K, Mg, and Zn. Examples of such compounds include compound Z,

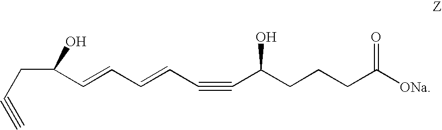

Other compounds suitable for use in methods of the invention include those of Formula 46,

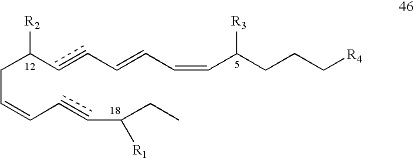

or a pharmaceutically acceptable salt or prodrug thereof, wherein:
each [symbol] independently designates a double or triple bond;
R¹, R², and R³ are each independently OR, OX¹, SR, SX², N(R)₂, NHX³, NRC(O)R, NRC(O)N(R)₂, C(O)OR, C(O)N(R)₂, SO₂R, NRSO₂R, C(O)R, or SO₂N(R)₂;
each R is independently selected from hydrogen or an optionally substituted group selected from C₁₋₆ aliphatic, a 3-8 membered saturated, partially unsaturated, or aryl ring having 0-4 heteroatoms independently selected from nitrogen, oxygen, or sulfur, or;
two R on the same nitrogen are taken together with the nitrogen to form a 5-8 membered heterocyclyl or heteroaryl ring having 1-3 heteroatoms independently selected from nitrogen, oxygen, or sulfur;
each X¹ is independently a suitable hydroxyl protecting group;
each X² is independently a suitable thiol protecting group;
each X³ is independently a suitable amino protecting group; and
R⁴ is NRC(O)R, NRC(O)N(R)₂, C(O)OR, C(O)N(R)₂, SO₂R, NRSO₂R, C(O)R, or SO₂N(R)₂.

Other compounds suitable for use in methods of the invention include those of Formula 47:

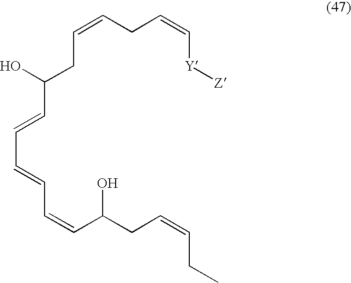

or a pharmaceutically acceptable salt or prodrug thereof, wherein:
Y′ is a bond or a linker selected from a ring containing up to 20 atoms or a chain of up to 20 atoms, provided that Y′ can include one or more nitrogen, oxygen, sulfur or phosphorous atoms, further provided that Y′ can include one or more substituents independently selected from hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, or sulfonyl, further provided that Y′ can contain one or more fused carbocyclic, heterocyclic, aryl or heteroaryl rings;
Z′ is selected from —CN, —C(NH)N(R″)(R″), —C(S)-A′, —C(S)R″, —C(O)-A′, —C(O)—R″, —C(O)—SR″, —C(O)—NH—S(O)₂—R″, —S(O)₂-A′, —S(O)₂—R″, S(O)₂N(R″)(R″), —P(O)₂-A′, —PO(OR″)-A′, -tetrazole, alkyltetrazole, or —CH₂OH, wherein A′ is selected from —OR″, —N(R″)(R″) or —OM′;
   each R″ is independently selected from hydrogen, alkyl, aryl, arylalkyl, heteroaryl, heteroarylalkyl or a detectable label molecule, wherein any alkyl-, aryl- or heteroaryl-containing moiety is optionally substituted with up to 3 independently selected substituents; and
   M′ is a cation.

In certain embodiments, a compound of formula 47 is represented by formula 48,

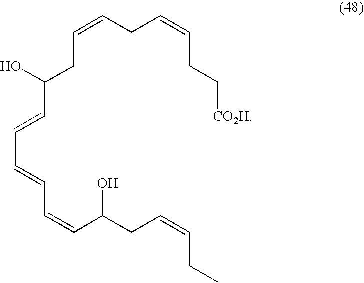

In certain embodiments, a compound of formula 47 is represented by formula 49,

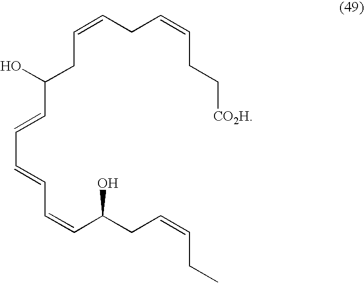

The compounds above (e.g., compounds of formula A or formulae 1 to 49) are known to be useful in the treatment or prevention of inflammation or inflammatory disease. Examples of such compounds are disclosed in the following patents and applications: US 2003/0191184, WO 2004/014835, WO 2004/078143, U.S. Pat. No. 6,670,396, US 2003/0236423, US 2005/0228047, US 2005/0238589 and US2005/0261255. These compounds are suitable for use in methods of the present invention.

Other compounds useful in this invention are compounds that are chemically similar variants to any of the compounds of formula A or formulae 1-49 set forth above. The term “chemically similar variants” includes, but is not limited to, replacement of various moieties with known biosteres; replacement of the end groups of one of the compounds above with a corresponding end group of any other compound above, modification of the orientation of any double bond in a compound, the replacement of any double bond with a triple bond in any compound, and the replacement of one or more substituents present in one of the compounds above with a corresponding substituent of any other compound.

Lipoxin compounds suitable for use in this invention include those of formula 50:

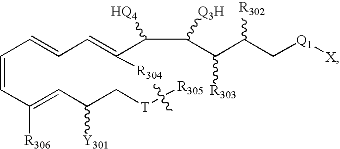

wherein:
X is R₃₀₁, OR₃₀₁, or SR₃₀₁;
R₃₀₁ is
(a) a hydrogen atom;
(b) an alkyl of 1 to 8 carbons atoms, inclusive, which may be straight chain or branched;
(c) a cycloalkyl of 3 to 10 carbon atoms;
(d) an aralkyl of 7 to 12 carbon atoms;
(e) phenyl;
(f) substituted phenyl

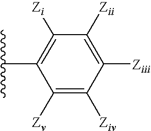

wherein ZᵢZᵢᵢ, Zᵢᵢᵢ, Zᵢᵥ and Zᵥ are each independently selected from —NO₂, —CN, —C(═O)—R₃₀₁, —SO₃H, a hydrogen atom, halogen, methyl, —ORₓ, wherein Rₓ is 1 to 8 carbon atoms, inclusive, which may be a straight chain or branched, and hydroxyl, wherein when any of ZᵢZᵢᵢ, Zᵢᵢᵢ, Zᵢᵥ or Zᵥ is C(═O)—R₃₀₁, said ZᵢZᵢᵢ, Zᵢᵢᵢ, Zᵢᵥ or Zᵥ is not substituted with another C(═O)—R₃₀₁.
(g) a detectable label molecule; or
(h) a straight or branched chain alkenyl of 2 to 8 carbon atoms, inclusive; Q₁ is (C═O), SO₂ or (CN), provided when Q₁ is CN, then X is absent;
Q₃ and Q₄ are each independently O, S or NH; one of R₃₀₂ and R₃₀₃ is a hydrogen atom and the other is:
   (a) H;
   (b) an alkyl of 1 to 8 carbon atoms, inclusive, which may be a straight chain or branched;
   (c) a cycloalkyl of 3 to 6 carbon atoms, inclusive;
   (d) an alkenyl of 2 to 8 carbon atoms, inclusive, which may be straight chain or branched; or
   (e) RₖQ₂R₁ wherein Q₂ is —O— or —S—; wherein Rₖ is alkylene of 0 to 6 carbons atoms, inclusive, which may be straight chain or branched and wherein R₁ is alkyl of 0 to 8 carbon atoms, inclusive, which may be straight chain or branched, provided when R₁ is 0, then R₁ is a hydrogen atom;
   R₃₀₄ is
   (a) H;
   (b) an alkyl of 1 to 6 carbon atoms, inclusive, which may be a straight chain or branched;
   R₃₀₅ is

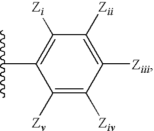

wherein ZᵢZᵢᵢ, Zᵢᵢᵢ, Zᵢᵥ and Zᵥ are defined as above;
R₃₀₆ is
(a) H;
(b) an alkyl from 1 to 4 carbon atoms, inclusive, straight chain or branched;
wherein Y₃₀₁ is —OH, methyl, —SH, an alkyl of 2 to 4 carbon atoms, inclusive, straight chain or branched, an alkoxy of 1 to 4 carbon atoms, inclusive, or (CH)ₚ(Z)_{q}, where p+q=3, p=0 to 3, q=0 to 3 and Z is cyano, nitro or a halogen; and
T is O or S, and pharmaceutically acceptable salts thereof.

Lipoxin compounds suitable for use in this invention include those of formulae 51, 52, 53 or 54:

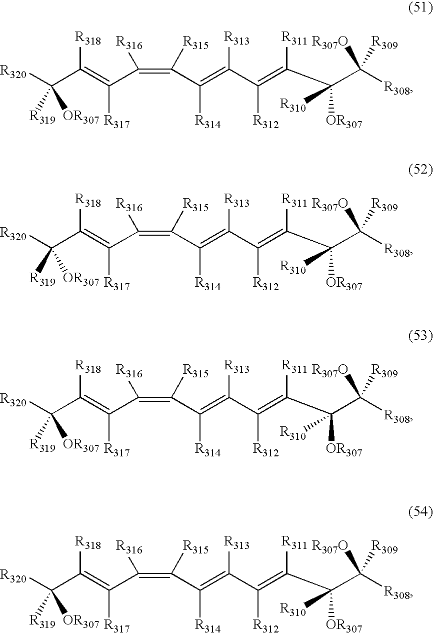

wherein:
each R₃₀₇ is independently selected from hydrogen and straight, branched, cyclic, saturated, or unsaturated alkyl having from 1 to 20 carbon atoms;
R₃₀₈, R₃₀₉, R₃₁₀, R₃₁₉, and R₃₂₀ are independently selected from:
(a) hydrogen;
(b) straight, branched, cyclic, saturated, or unsaturated alkyl having from 1 to 20 carbon atoms;
(c) substituted alkyl having from 1 to 20 carbon atoms, wherein the alkyl is substituted with one or more substituents selected from halo, hydroxy, lower alkoxy, aryloxy, amino, alkylamino, dialkylamino, acylamino, arylamino, hydroxyamino, alkoxyamino, alkylthio, arylthio, carboxy, carboxamido, carboalkoxy, aryl, and heteroaryl;
(d) substituted aryl or heteroaryl, wherein the aryl or heteroaryl is substituted with one or more substituents selected from alkyl, cycloalkyl, alkoxy, halo, aryl, heteroaryl, carboxyl, and carboxamido; and
(e) Z-Y, wherein:
   Z is selected from a straight, branched, cyclic, saturated, or unsaturated alkyl having from 1 to 20 carbon atoms; substituted lower alkyl, wherein the alkyl is substituted with one or more substituents selected from halo, hydroxy, lower alkoxy, aryloxy, amino, alkylamino, dialkylamino, acylamino, arylamino, hydroxyamino, alkoxyamino, alkylthio, arylthio, carboxy, carboxamido, carboalkoxy, aryl, and heteroaryl; and substituted aryl or heteroaryl, wherein the aryl or heteroaryl is substituted with one or more substituents selected from alkyl, cycloalkyl, alkoxy, halo, aryl, heteroaryl, carboxyl, and carboxamido; and
   Y is selected from hydrogen; alkyl; cycloalkyl; carboxyl; carboxamido; aryl; heteroaryl; substituted aryl or heteroaryl, wherein the aryl or heteroaryl is substituted with one or more substituents selected from alkyl, cycloalkyl, alkoxy, halo, aryl, heteroaryl, carboxyl, and carboxamido; and

R₃₁₁ to R₃₁₈ are independently selected from:
(a) hydrogen;
(b) halo;
(c) straight, branched, cyclic, saturated, or unsaturated alkyl having from 1 to 20 carbon atoms;
(d) substituted alkyl having from 1 to 20 carbon atoms, wherein the alkyl is substituted with one or more substituents selected from halo, hydroxy, lower alkoxy, aryloxy, amino, alkylamino, dialkylamino, acylamino, arylamino, hydroxyamino, alkoxyamino, alkylthio, arylthio, carboxy, carboxamido, carboalkoxy, aryl, and heteroaryl;
(e) substituted aryl or heteroaryl, wherein the aryl or heteroaryl is substituted with one or more substituents selected from alkyl, cycloalkyl, alkoxy, halo, aryl, heteroaryl, carboxyl, and carboxamido; or

R₃₀₈ to R₃₂₀ are independently a bond that forms a carbon-carbon double bond, a carbon-carbon triple bond, or a ring with the lipoxin backbone; or any two of R₃₀₇ to R₃₂₀ are taken together with the atoms to which they are bound and optionally to 1 to 6 oxygen atoms, 1 to 6 nitrogen atoms, or both 1 to 6 oxygen atoms and 1 to 6 nitrogen atoms, to form a ring containing 3 to 20 atoms.

Lipoxin compounds suitable for use in this invention include those of formula 55:

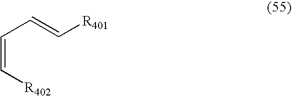

wherein:

R₄₀₀ is selected from:

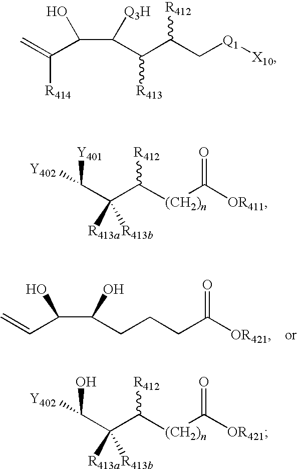

R₄₀₂ is selected from:

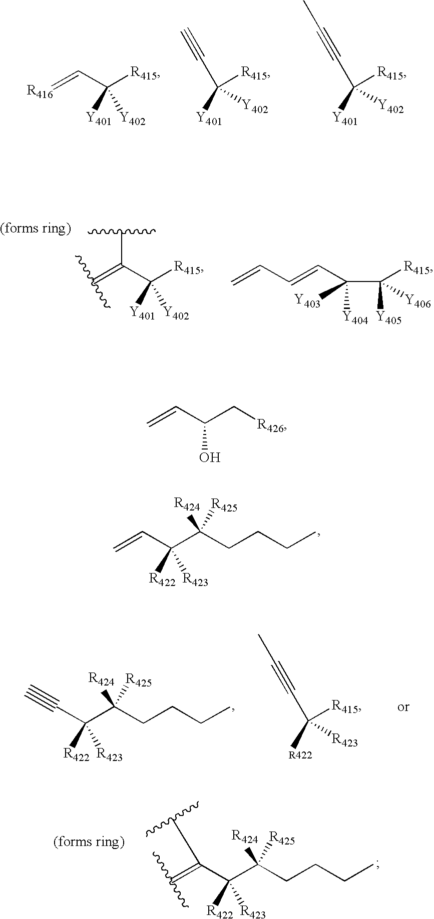

X₁₀ is R₄₁₁, OR₄₁₁, or SR₄₁₁;

R₄₁₁ is
(a) a hydrogen atom;
(b) an alkyl of 1 to 8 carbons atoms, inclusive, which may be straight chain or branched;
(c) a cycloalkyl of 3 to 10 carbon atoms;
(d) an aralkyl of 7 to 12 carbon atoms;
(e) phenyl;
(f) substituted phenyl

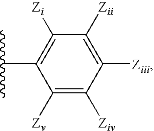

wherein ZᵢZᵢᵢ, Zᵢᵢᵢ, Zᵢᵥ and Zᵥ are each independently selected from —NO₂, —CN, —C(═O)—R₄₁₁, —SO₃H, a hydrogen atom, halogen, methyl, —ORₓ, wherein Rₓ is 1 to 8 carbon atoms, inclusive, which may be a straight chain or branched, and hydroxyl; wherein when any of ZᵢZᵢᵢ, Zᵢᵢᵢ, Zᵢᵥ or Zᵥ is C(═O)—R₄₁₁, said ZᵢZᵢᵢ, Zᵢᵢᵢ, Zᵢᵥ or Zᵥ is not substituted with another C(═O)—R₄₁₁.
(g) a detectable label molecule; or
(h) a straight or branched chain alkenyl of 2 to 8 carbon atoms, inclusive;

Q₁ is (C═O), SO₂ or (CN);

Q₃ is O, S or NH;

one of R₄₁₂ and R₄₁₃ is a hydrogen atom and the other is selected from:
(a) H;
(b) an alkyl of 1 to 8 carbon atoms, inclusive, which can be straight chain or branched;
(c) a cycloalkyl of 3 to 6 carbon atoms, inclusive;
(d) an alkenyl of 2 to 8 carbon atoms, inclusive, which can be straight chain or branched; or
(e) R₄₃₁Q₂R₄₃₂ wherein Q₂ is —O— or —S—; wherein R₄₃₁ is alkylene of 0 to 6 carbons atoms, inclusive, which can be straight chain or branched and wherein R₄₃₁ is alkyl of 0 to 8 carbon atoms, inclusive, which can be straight chain or branched;

R₄₁₃ₐ and R_{413b} are each independently:
(a) H;
(b) an alkyl of 1 to 8 carbon atoms, inclusive, which can be straight chain or branched;
(c) a cycloalkyl of 3 to 6 carbon atoms, inclusive;
(d) an alkenyl of 2 to 8 carbon atoms, inclusive, which can be straight chain or branched; or
(e) R₄₃₁Q₂R₄₃₂ wherein R₄₃₁, Q₂, and R₄₃₂ are as defined above;

R₄₁₄ is
(a) H;
(b) an alkyl of 1 to 6 carbon atoms, inclusive, can be straight chain or branched;

R₄₁₅ is
(a) an alkyl of 1 to 9 carbon atoms which can be straight chain or branched;
(b) —(CH₂)—Rᵢ

wherein n=0 to 4 and Rᵢ is
(i) a cycloalkyl of 3 to 10 carbon atoms, inclusive;
(ii) a phenyl; or
(iii) substituted phenyl

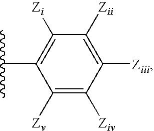

wherein Zᵢ through Zᵥ are as defined above;
(b) R₄₃₁Q₂R₄₃₂, wherein R₄₃₁, Q₂, and R₄₃₂ are as defined above;
(c) —C(Rᵢᵢᵢ)(Rᵢᵥ)—Rᵢ,

wherein Rᵢᵢᵢ and Rᵢᵥ are each independently:
(i) a hydrogen atom;
(ii) (CH)ₚ(Z)_{q}, wherein Z, p, and q are as defined above;
(e) a haloalkyl of 1 to 8 carbon atoms, inclusive, and 1 to 6 halogen atoms, inclusive, straight chain or branched;

R₄₁₆ is
(a) H;
(b) an alkyl from 1 to 4 carbon atoms, inclusive, straight chain or branched;
(c) a halogen;

one of Y₄₀₁ or Y₄₀₂ is —OH, methyl, or —SH, and wherein the other is selected from:
(a) H;
(b) (CH)ₚ(Z)_{q} where p+q=3, p=0 to 3, q=0 to 3 and each Z, independently, is cyano, nitro or a halogen;
(c) an alkyl of 2 to 4 carbon atoms, inclusive, straight chain or branched; or
(d) an alkoxy of 1 to 4 carbon atoms, inclusive,

or Y₄₀₁ and Y₄₀₂ taken together are:
(d)═NH; or
(e)═O;

one of Y₄₀₃ or Y₄₀₄ is —OH, methyl, or —SH, and wherein the other is selected from:
(a) H;
(b) (CH)ₚ(Z)_{q} wherein Z, p, and q are as defined above;
(c) an alkyl of 2 to 4 carbon atoms, inclusive, straight chain or branched; or
(d) an alkoxy of 1 to 4 carbon atoms, inclusive,

or Y₄₀₁ and Y₄₀₂ taken together are:
(a) ═NH; or
(b) ═O;

one of Y₄₀₅ or Y₄₀₆ is —OH, methyl, or —SH, and wherein the other is selected from:
(a) H
(b) (CH)ₚ(Z)_{q} wherein Z, p, and q are as defined above;
(c) an alkyl of 2 to 4 carbon atoms, inclusive, straight chain or branched; or
(d) an alkoxy of 1 to 4 carbon atoms, inclusive,

or Y₄₀₁ and Y₄₀₂ taken together are:
(a) ═NH; or
(b) ═O;

R₄₂₁ is
(a) H; or
(b) alkyl of 1 to 8 carbon atoms;

R₄₂₂ and R₄₂₃ are each independently:
(a) H;
(b) a hydroxyl, or a thiol;
(c) a methyl or a halomethyl;
(d) a halogen; or
(e) an alkoxy of 1 to 3 carbon atoms;

R₄₂₄ and R₄₂₅ are each independently:
(a) H;
(b) a hydroxyl, or a thiol;
(c) a methyl or a halomethyl;
(d) a halogen;
(e) an alkoxy of 1 to 3 carbon atoms; or
(f) an alkyl or haloalkyl of 2 to 4 carbon atoms inclusive, which can be straight chain or branched; and

R₄₂₆ is
(a) a substituted phenyl

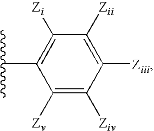

wherein Zᵢ through Zᵥ are as defined above;
(b) a substituted phenoxy

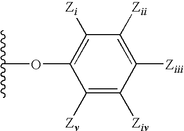

wherein Zᵢ through Zᵥ are as defined above; or
(c)

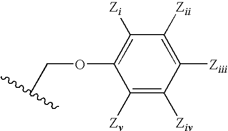

wherein Zᵢ through Zᵥ are as defined above.

Lipoxin compounds suitable for use in this invention include those of formula 56:

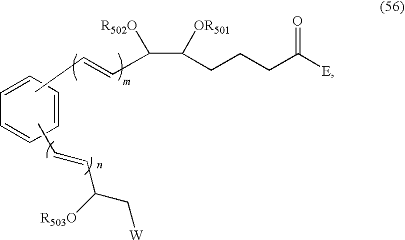

wherein:
E is hydroxy, alkoxy, aryloxy, amino, alkylamino, dialkylamino or —OM, where M is a cation selected from ammonium, tetra-alkyl ammonium, and the cations of sodium, potassium, magnesium and zinc;
W is hydrogen, alkyl, alkenyl, alkynyl, aryl, heteroaryl, halo, hydroxy, alkoxy, aryloxy, carboxy, amino, alkylamino, dialkylamino, acylamino, carboxamido, or sulfonamide;
each of R₅₀₁-R₅₀₃ are independently selected from hydrogen, alkyl, aryl, acyl or alkoxyacyl;
n is 0, 1 or 2;
m is 1 or 2; and
the two substituents on the phenyl ring are ortho, meta, or para.

Lipoxin compounds suitable for use in this invention include those of formula 57:

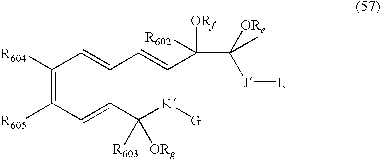

wherein:
I is selected from: —C(O)-E, —SO₂-E, —PO(OR)-E, where E is hydroxy, alkoxy, aryloxy, amino, alkylamino, dialkylamino, or —OM, where M is a cation selected from ammonium, tetra-alkyl ammonium, Na, K, Mg, and Zn; and R is hydroxyl or alkoxy
J′ and K′ are linkers independently selected from a chain of up to 20 atoms and a ring containing up to 20 atoms, provided that J′ and K′ can independently include one or more nitrogen, oxygen, sulfur or phosphorous atoms, and further provided that J′ and K′ can independently include one or more substituents selected from hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, and sulfonyl, and further provided that J′ and K′ can also contain one or more fused carbocyclic, heterocyclic, aryl or heteroaryl rings, and provided that linkers J′ and K′ are connected to the adjacent C(R)OR group via a carbon atom or a C-heteroatom bond where the heteroatom is oxygen, sulfur, phosphorous or nitrogen;
G is selected from hydrogen, alkyl, alkenyl, alkynyl, aryl, heteroaryl, chloro, iodo, bromo, fluoro, hydroxy, alkoxy, aryloxy, carboxy, amino, alkylamino, dialkylamino, acylamino, and carboxamido.
Re, Rf and Rg, are independently selected from hydrogen, alkyl, aryl, heteroaryl, acyl, silyl, alkoxyacyl and aminoacyl;

R₆₀₁, R₆₀₂ and R₆₀₃ are independently selected from hydrogen, alkyl, aryl and heteroaryl, provided that R₆₀₁, R₆₀₂ and R₆₀₃ can independently be connected to linkers J′ or K′;

R₆₀₄ and R₆₀₅ are independently selected from hydrogen, alkyl, alkenyl, alkynyl, aryl, heteroaryl, fluoro, and provided that R₆₀₄ and R₆₀₅ can be joined together to form a carbocyclic, heterocyclic or aromatic ring, and further provided that R₆₀₄ and R₆₀₅ can be replaced by a bond to form a triple bond.

Other compounds suitable for use in methods of the invention are the oxylipins described in international applications WO 2006055965, WO 2007090162, and WO 2008/103753, the compounds in which are incorporated herein by reference. Examples of such compounds are those of formulae 58-132, as shown in Table 1. These compounds include long chain omega-6 fatty acids, docosapentaenoic acid (DPAn-6) (compounds 58-73) and docosatetraenoic acid (DTAn-6) (compounds 74-83), and the omega-3 counterpart of DPAn-6, docosapentaenoic acid (DPAn-3) (compounds 84-97). Further compounds are the docosanoids 98-115, the γ-linolenic acids (GLA) (compounds 116-122), and the stearidonic acids (SDA) (compounds 123-132).

TABLE 1

| | |
|---|---|
| 10,17-Dihydroxy DPAn-6 (58) | 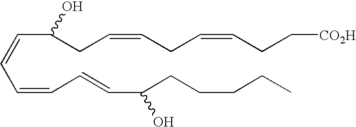 |
| 16,17-Dihydroxy DPAn-6 (59) | 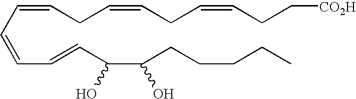 |
| 4,5-Dihydroxy DPAn-6 (60) | 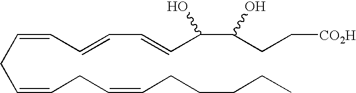 |
| 7,17-Dihydroxy DPAn-6 (61) | 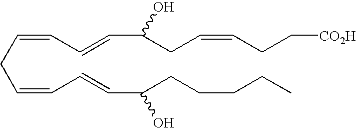 |
| 7-Hydroxy DPAn-6 (62) | 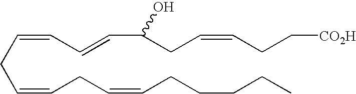 |
| 10-hydroxy DPAn-6 (63) | 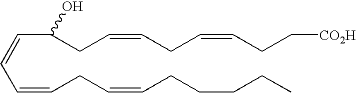 |
| 13-Hydroxy DPAn-6 (64) | 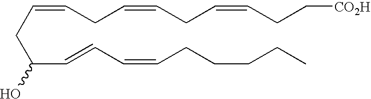 |
| 17-hydroxy DPAn-6 (65) | 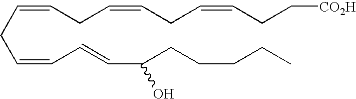 |
| 4,5,17-Trihydroxy DPAn-6 (66) | 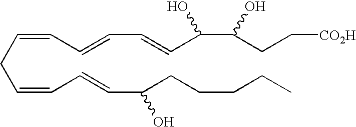 |
| 7,16,17-Trihydroxy DPAn-6 (67) | 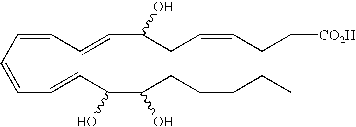 |
| 8-Hydroxy DPAn-6 (68) | 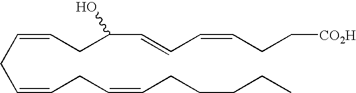 |
| 14-Hydroxy DPAn-6 (69) | 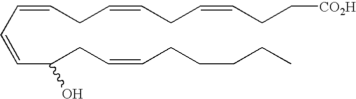 |
| 13,17-Dihydroxy DPAn-6 (70) | 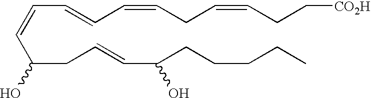 |
| 7,14-Dihydroxy DPAn-6 (71) | 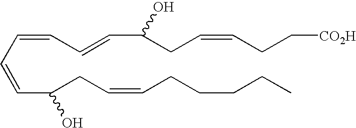 |
| 8,14-Dihydroxy DPAn-6 (72) | 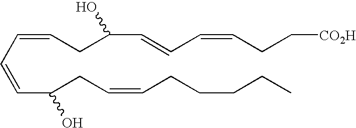 |
| 11-Hydroxy DPAn-6 (73) | 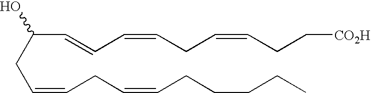 |
| 10,17-Dihydroxy-DTAn-6 (74) | 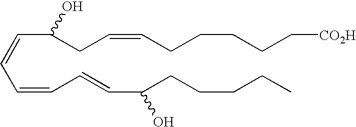 |
| 16,17-Dihydroxy-DTAn-6 (75) | 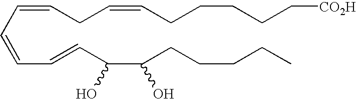 |
| 4,5-Dihydroxy-DTAn-6 (76) | 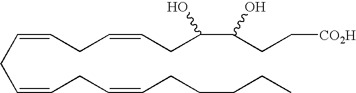 |
| 7,17-Dihydroxy-DTAn-6 (77) | 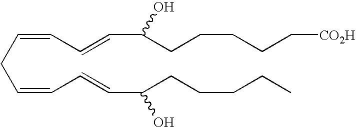 |
| 7-Hydroxy-DTAn-6 (78) | 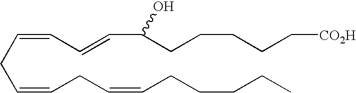 |
| 10-Hydroxy-DTAn-6 (79) | 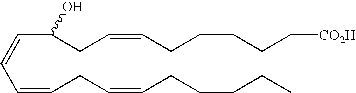 |
| 13-Hydroxy-DTAn-6 (80) | 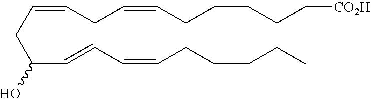 |
| 17-Hydroxy-DTAn-6 (81) | 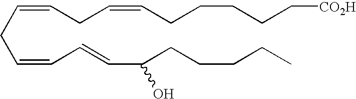 |
| 4,5,17-Trihydroxy-DTAn-6 (82) | 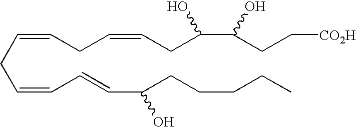 |
| 7,16,17-Trihydroxy-DTAn-6 (83) | 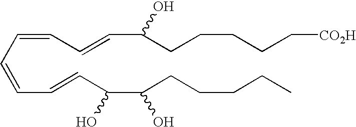 |
| 10,17-Dihydroxy DPAn-3 (84) | 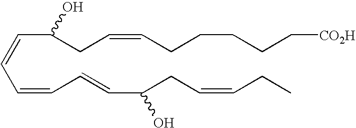 |
| 10,20-Dihydroxy DPAn-3 (85) | 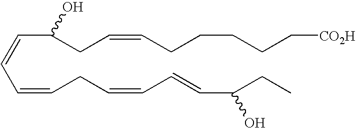 |
| 13,20-Dihydroxy DPAn-3 (86) | 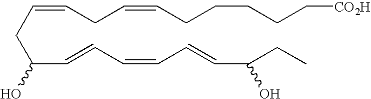 |
| 16,17-Dihydroxy DPAn-3 (87) | 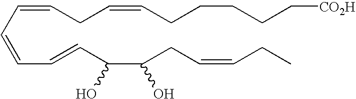 |
| 7,17-Dihydroxy DPAn-3 (88) | 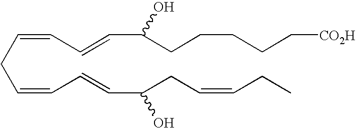 |
| 7-Hydroxy DPAn-3 (89) | 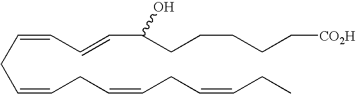 |
| 10-Hydroxy DPAn-3 (90) | 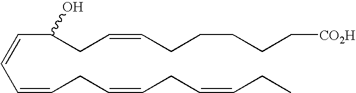 |
| 13-Hydroxy DPAn-3 (91) | 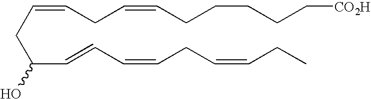 |
| 17-Hydroxy DPAn-3 (92) | 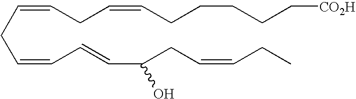 |
| 7,16,17-Trihydroxy DPAn-3 (93) | 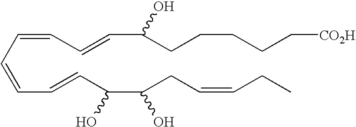 |
| 16-Hydroxy DPAn-3 (94) | 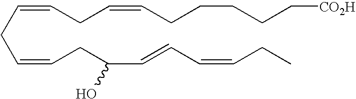 |
| 11-Hydroxy DPAn-3 (95) | 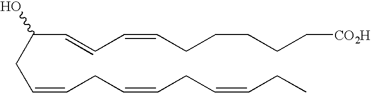 |
| 14-Hydroxy DPAn-3 (96) | 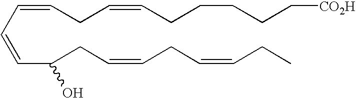 |
| 8,14-Dihydroxy DPAn-3 (97) | 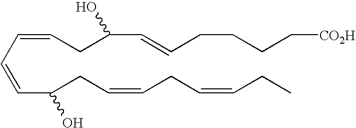 |
| 10,11-Epoxy DHA (98) | 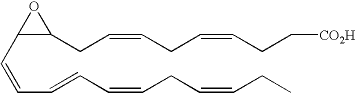 |
| 13,14-Dihydroxy DHA (99) | 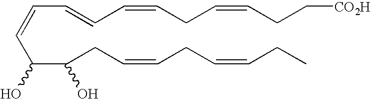 |
| 13,14-Epoxy DHA (100) | 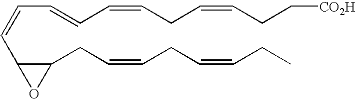 |
| 19,20-Epoxy DHA (101) | 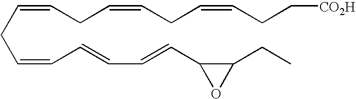 |
| 7,8-Epoxy DHA (102) | 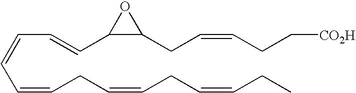 |
| 4,5-Epoxy-17-OH DPA (103) | 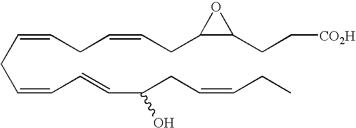 |
| 7,16,17-Trihydroxy DTAn-3 (104) | 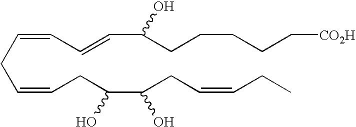 |
| 16,17-Dihidroxy DTAn-3 (105) | 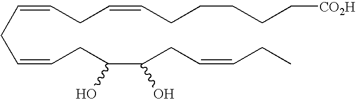 |
| 10,16,17-Trihydroxy DTRAn-6 (106) | 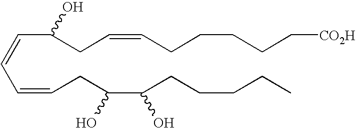 |
| 16,17-Dihydroxy DTRAn-6 (107) | 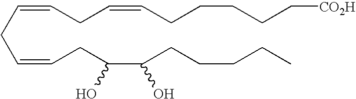 |
| 7,16,17-Trihydroxy DTRAn-6 (108) | 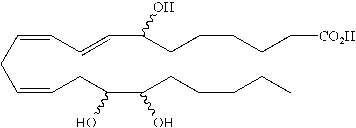 |
| 15-epi-lipoxin A4 (109) | 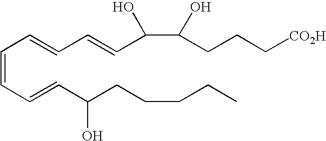 |
| 16,17-epoxy DHA (110) | 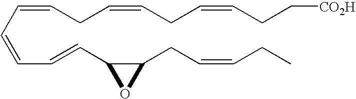 |
| 7,8-epoxy DPA (111) | 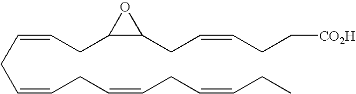 |
| 10,11 epoxy DPA (112) | 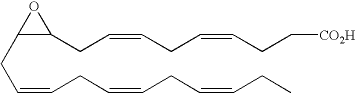 |
| 19,20 epoxy DPA (113) | 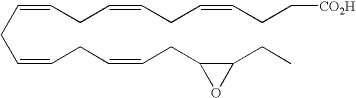 |
| 7-hydroxy DHA (114) | 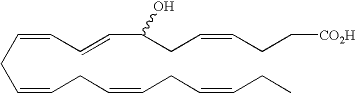 |
| 13,14 epoxy DPA (115) | 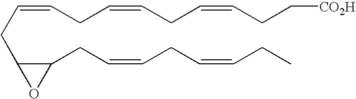 |
| 6-hydroxy GLA (116) | 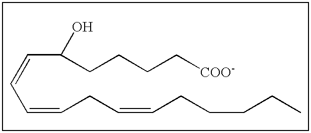 |
| 10-hydroxy GLA (117) | 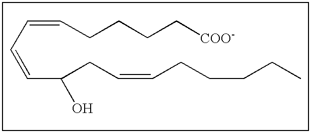 |
| 7-hydroxy GLA (118) | 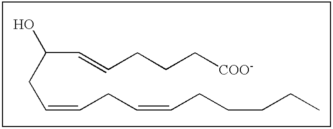 |
| 12-hydroxy GLA (119) | 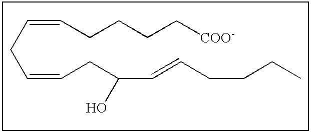 |
| 9-hydroxy GLA (120) | 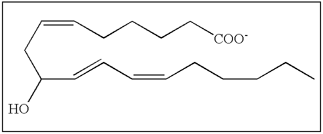 |
| 13-hydroxy GLA (121) | 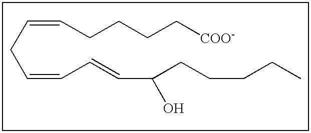 |
| 6,13 dihydroxy GLA (122) | 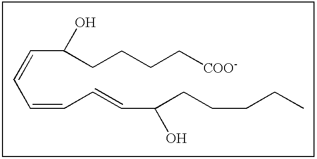 |
| 6-hydroxy SDA (123) | 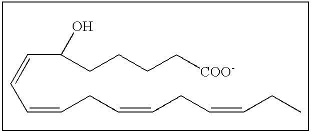 |
| 10-hydroxy SDA (124) | 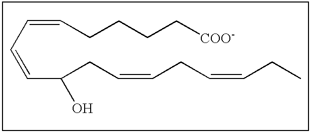 |
| 7-hydroxy SDA (125) | 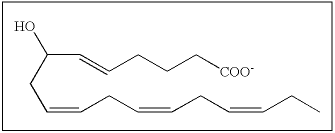 |
| 12-hydroxy SDA (126) | 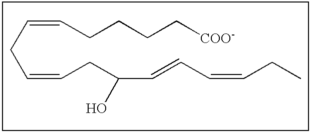 |
| 9-hydroxy SDA (127) | 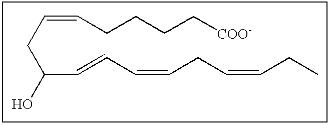 |
| 13-hydroxy SDA (128) | 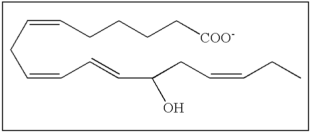 |
| 15-hydroxy SDA (129) | 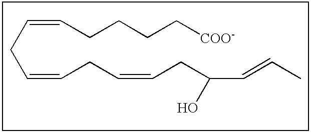 |
| 16-hydroxy SDA (130) | 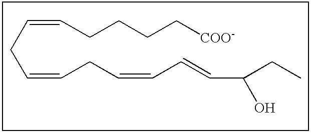 |
| 6,13 dihydroxy SDA (131) | 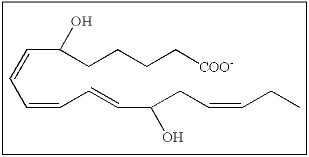 |
| 6,16 dihydroxy SDA (132) | 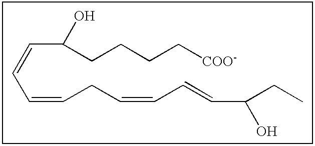 |

Other oxylipin compounds that are suitable for use in methods of the invention include analogs of the compounds shown in Table 1. Such compounds include but are not limited to those analogs wherein one or more double bonds are replaced by triple bonds, those wherein one or more carboxy groups are derivatized to form esters, amides or salts, those wherein the hydroxyl-bearing carbons are further derivatized (with, for example, a substituted or unsubstituted, branched or unbranched alkyl, alkenyl, or alkynyl group, substituted or unsubstituted aryl group, substituted or unsubstituted, branched or unbranched alkylaryl group, halogen atom) to form tertiary alcohols (or ethers, esters, or other derivatives thereof), those wherein one or more hydroxyl groups are derivatized to form esters or protected alcohols, or those having combinations of any of the foregoing modifications.

Further oxylipin compounds suitable for use in methods of the invention include the following: isolated docosanoids of docosapentaenoic acid (DPAn-6); monohydroxy, dihydroxy, and trihydroxy derivatives of DPAn-6; isolated docosanoids of docosapentaenoic acid (DPAn-3); monohydroxy, dihydroxy, and trihydroxy derivatives of DPAn-3; isolated docosanoids of docosapentaenoic acid (DTAn-6); or monohydroxy, dihydroxy, and trihydroxy derivatives of DTAn-6.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group, preferably a lower alkyl group, having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. Likewise, preferred cycloalkyls have from 3-10 carbon atoms in their ring structure, and more preferably have 5, 6 or 7 carbons in the ring structure.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS-.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

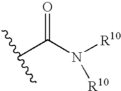

wherein each R¹⁰ independently represent a hydrogen or hydrocarbyl group, or two R¹⁰ are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

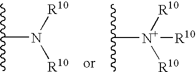

wherein each R¹⁰ independently represents a hydrogen or a hydrocarbyl group, or two R¹⁰ are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

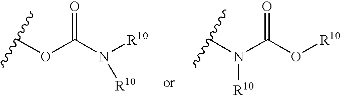

wherein each R¹⁰ independently represent hydrogen or a hydrocarbyl group, or both R¹⁰ groups taken together with the intervening atom(s) complete a heterocycle having from 4 to 8 atoms in the ring structure.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—R¹⁰, wherein R¹⁰ represents a hydrocarbyl group.

The term “carboxy”, as used herein, refers to a group represented by the formula —CO₂H.

The term “ester”, as used herein, refers to a group —C(O)OR¹⁰ wherein R¹⁰ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

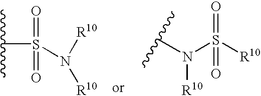

wherein each R¹⁰ independently represents hydrogen or hydrocarbyl, or both R¹⁰ groups taken together with the intervening atom(s) complete a heterocycle having from 4 to 8 atoms in the ring structure.

The term “sulfoxide” is art-recognized and refers to the group —S(O)—R¹⁰, wherein R¹⁰ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R¹⁰, wherein R¹⁰ represents a hydrocarbyl.

The term “thioalkyl”, as used herein, refers to an alkyl group substituted with a thiol group.

The term “thioester”, as used herein, refers to a group —C(O)SR¹⁰ or —SC(O)R¹⁰ wherein R¹⁰ represents a hydrocarbyl.

The term “thioether”, as used herein, is equivalent to an ether, wherein the oxygen is replaced with a sulfur.

The term “urea” is art-recognized and may be represented by the general formula

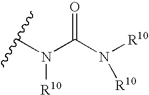

wherein each R¹⁰ independently represent hydrogen or a hydrocarbyl, or two occurrences of R¹⁰ taken together with the intervening atom(s) complete a heterocycle having from 4 to 8 atoms in the ring structure.

The term “prodrug” is intended to encompass compounds which, under physiologic conditions, are converted into the therapeutically active agents of the present invention (e.g., a compound of formula A or formulae 1-49, a lipoxin compound, or an oxylipin compound). A common method for making a prodrug is to include one or more selected moieties which are hydrolyzed under physiologic conditions to reveal the desired molecule. In other embodiments, the prodrug is converted by an enzymatic activity of the host animal. For example, esters (e.g., esters of alcohols or carboxylic acids) are preferred prodrugs of the present invention. In certain embodiments, some or all of the compounds of formula A, compounds of any one of formulae 1-49, lipoxins, or oxylipins, all or a portion of a compound of formula A, compound of any one of formulae 1-49, lipoxin, or oxylipin in a formulation represented above can be replaced with the corresponding suitable prodrug, e.g., wherein a hydroxyl or carboxylic acid present in the parent compound is presented as an ester.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3rd Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPPS groups), glycol ethers, such as ethylene glycol and propylene glycol derivatives and allyl ethers.

The term “healthcare providers” refers to individuals or organizations that provide healthcare services to a person, community, etc. Examples of “healthcare providers” include doctors, hospitals, continuing care retirement communities, skilled nursing facilities, subacute care facilities, clinics, multispecialty clinics, freestanding ambulatory centers, home health agencies, and HMO's.

The term “treating” refers to: preventing a disease, disorder or condition from occurring in a cell, a tissue, a system, animal or human which may be predisposed to the disease, disorder and/or condition but has not yet been diagnosed as having it; stabilizing a disease, disorder or condition, i.e., arresting its development; and relieving one or more symptoms of the disease, disorder or condition, i.e., causing regression of the disease, disorder and/or condition.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, “immunosuppressive agent” refers to agents that suppress the body's ability to elicit an immunological response to the presence of an antigen/allergen. For example, the ability to fight off disease or reject a transplanted organ. Another term for these agents is anti-rejection agents. Not only are they are used to treat organ rejection after transplantation, but many other diseases of immunological etiology such as Crohn's disease, rheumatoid arthritis, lupus, multiple sclerosis, psoriasis, and other diseases and disorders as described herein.

The term “graft”, as used herein, refers to a body part, organ, tissue, or cells. Grafts may comprise all or part of one or more organs such as liver, kidney, heart or lung; body parts such as bone or skeletal matrix; tissue such as skin, intestines, endocrine glands; or progenitor stem cells of various types.

The synthesis of each of the compounds of formula A, compounds of any one of formulae 1-49, lipoxins, or oxylipins set forth above can be achieved by methods well-known in the art. For example, the synthesis of compounds of formula A or formulae 1-49 is set forth in US 2003/0191184, WO 2004/014835, WO 2004/078143, U.S. Pat. No. 6,670,396, US 2003/0236423 and US 2005/0228047, all of which are herein incorporated by reference. The synthesis of lipoxin compounds is set forth in US 2002/0107289, US 2004/0019110, US 2006/0009521, US 2005/0203184, US 2005/0113443, all of which are herein incorporated by reference. The preparation of oxylipin compounds is set forth in WO 2006/055965, WO 2007/090162, and WO 2008/103753, all of which are herein incorporated by reference.

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of formula A, compound of any one of formulae 1-49, lipoxin compound, oxylipin compound, or aspirin and/or an omega-3 fatty acid and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil or injectable organic esters. In a preferred embodiment, when such pharmaceutical compositions are for human administration, the aqueous solution is pyrogen free, or substantially pyrogen free. The excipients can be chosen, for example, to effect delayed release of an agent or to selectively target one or more cells, tissues or organs. The pharmaceutical composition can be in dosage unit form such as tablet, capsule, sprinkle capsule, granule, powder, syrup, suppository, injection or the like. The composition can also be present in a transdermal delivery system, e.g., a skin patch.

A pharmaceutically acceptable carrier can contain physiologically acceptable agents that act, for example, to stabilize or to increase the absorption of a compound such as a compound of formula A, compound of any one of formulae 1-49, lipoxin compound, oxylipin compound, or aspirin and/or an omega-3 fatty acid. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, boluses, powders, granules, pastes for application to the tongue); sublingually; anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein. The most preferred route of administration is the oral route.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of formula A, compound of any one of formulae 1-49, lipoxin compound, oxylipin compound, or aspirin and/or an omega-3 fatty acid, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatable with such fluids.

Formulations of the present invention can be administered in a manner generally known to those skilled in the art. In certain embodiments, the formulation is administered using an eyedropper. The eyedropper can be constructed in any suitable way. It may be desirable to utilize a measured dose eyedropper of the type described within U.S. Pat. No. 5,514,118 or an illuminated eyedropper device of the type described in U.S. Pat. No. 5,584,823. A range of other eye droppers can also be utilized of the type described within the following U.S. Pat. Nos. 5,059,188; 4,834,727; 4,629,456; and 4,515,295. The patents cited here which disclose eyedroppers are incorporated herein by reference as are the various patents and publications cited and discussed within these patents.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsuled matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

In certain embodiments, the method of inhibiting immune function, suppressing an immune response, or treating an autoimmune disease or autoimmune disorder comprises conjointly administering a compound of formula A, compound of any one of formulae 1-49, lipoxin compound, oxylipin compound, or combination of aspirin and an omega-3 fatty acid conjointly with another therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. Thus, an individual who receives such treatment can benefit from a combined effect of different therapeutic compounds.

In certain embodiments, different compounds of formulae A, compounds of any one of formulae 1-49, lipoxin compounds, or oxylipin compounds may be conjointly administered with other agents suitable for modulating immune function, suppressing immune response, treating an autoimmune disease or autoimmune disorder, or treating a disease, sequela or pathological condition mediated by an activation of the immune system. For example, the following immunosuppressive agents may be conjointly administered with a compound of formula A, compound of any one of formulae 1-49, lipoxin compound, oxylipin compound, or combination of aspirin and an omega-3 fatty acid: cyclosporin, cyclosporin A, tacrolimus, rapamycin, everolimus, FK-506, cyclophosphamide, azathioprene, methotrexate, brequinar, leflunomide, mizoribine, mycophenolic acid, mycophenolate mofetil, 15-deoxyspergualine, triamcinolone acetonide, decadron, daclizumab, basiliximab, glatiramer acetate, infliximab, muromonab, octreotide, muramylic acid dipeptide derivatives, levamisole, niridazole, oxysuran, flagyl, and sirolimus.

In certain embodiments, different compounds of formulae A, compounds of any one of formulae 1-49, lipoxin compounds, or oxylipin compounds may be conjointly administered with one another. Moreover, such combinations may be conjointly administered with other therapeutic agents, such as other agents suitable for modulating immune function, suppressing immune response, treating an autoimmune disease or autoimmune disorder, or treating a disease, sequela or pathological condition mediated by an activation of the immune system, such as the agents identified above.

In embodiments where a combination of aspirin and an omega-3 fatty acid are administered, the aspirin and omega-3 fatty acid can be administered simultaneously, e.g., as a single formulation comprising both components or in separate formulations, or can be administered at separate times, provided that, at least at certain times during the therapeutic regimen, both the aspirin and omega-3 fatty acid are present simultaneously in the patient at levels that allow the omega-3 fatty acid to be metabolized as described in Serhan, et. al., 2002, J. Exp. Med., 196: 1025-1037. In certain such embodiments, the omega-3 fatty acid is provided in the form of a partially purified natural extract, such as fish oil, while in other embodiments, the omega-3 fatty acid may be provided as a substantially pure preparation of one or more omega-3 fatty acids, such as a C18:3, C20:5, or C22:6 fatty acid, particularly eicosapentaenoic acid or docosahexaenoic acid. A substantially pure preparation of one or more omega-3 fatty acids refers to a composition wherein the fatty acid component is at least 90%, at least 95%, or even at least 98% of one or more omega-3 fatty acids, such as one or more specified omega-3 fatty acids. Non-fatty acid components, such as excipients or other materials added during formulation, are not considered for the purpose of determining whether the fatty acid component meets the desired level of purity.

In certain embodiments, a COX-2 inhibitor other than aspirin, such as celecoxib, rofecoxib, valdecoxib, lumiracoxib, etoricoxib, NS-398, or parecoxib, may be used in combination with an omega-3 fatty acid for modulating immune function, suppressing immune response, treating autoimmune diseases or autoimmune disorders, or treating diseases, sequelae or pathological conditions mediated by an activation of the immune system in any of the various embodiments discussed herein. In certain embodiments, a non-selective NSAID other than aspirin, such as diclofenac, diflunisal, etodolac, fenoprofen, ibuprofen, indomethacin, ketoprofen, ketorolac, mefenamic acid, meloxicam, nabumetone, naproxen, oxaprozin, piroxicam, salsalate, sulindac, or tolmetin, may be used in combination with an omega-3 fatty acid for modulating immune function, suppressing immune response, treating autoimmune diseases or autoimmune disorders, or treating diseases, sequelae or pathological conditions mediated by an activation of the immune system in any of the various embodiments discussed herein. The combination of different COX-2 inhibitors or non-selective NSAIDs with an omega-3 fatty acid may result in the production of different subsets or proportions of active omega-3 metabolites.

This invention includes the use of pharmaceutically acceptable salts of compounds of formula A, compounds of any one of formulae 1-49, lipoxin compounds, or oxylipin compounds in the compositions and methods of the present invention. In certain embodiments, contemplated salts of the invention include alkyl, dialkyl, trialkyl or tetra-alkyl ammonium salts. In certain embodiments, contemplated salts of the invention include Na, Ca, K, Mg, Zn or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The present invention provides a kit comprising:
a) a pharmaceutical formulation comprising a compound of formula A, a compound of any one of formulae 1-49, a lipoxin compound, an oxylipin compound, or a combination of aspirin and an omega-3 fatty acid; and
b) instructions for the administration of the pharmaceutical formulation for modulating immune function, suppressing immune response, treating an autoimmune disease or autoimmune disorder, or treating a disease, sequela or pathological condition mediated by an activation of the immune system.

The present invention provides a kit comprising:
a) one or more single dosage forms each comprising a compound of formula A, a compound of any one of formulae 1-49, a lipoxin compound, an oxylipin compound, or a combination of aspirin and an omega-3 fatty acid and a pharmaceutically acceptable excipient; and
b) instructions for administering the single dosage forms for modulating immune function, suppressing immune response, treating an autoimmune disease or autoimmune disorder, or treating a disease, sequela or pathological condition mediated by an activation of the immune system.

In certain embodiments, the present invention provides a kit comprising:
a) one or more single dosage forms each comprising a dose of a compound of formula A, a compound of any one of formulae 1-49, a lipoxin compound, an oxylipin compound, or a combination of aspirin and an omega-3 fatty acid;
b) one or more single dosage forms of a second agent suitable for modulating immune function, suppressing immune response, treating an autoimmune disease or autoimmune disorder, or treating a disease, sequela or pathological condition mediated by an activation of the immune system as mentioned above; and
c) instructions for the administration of the compound of formula A, compound of any one of formulae 1-49, lipoxin compound, oxylipin compound, or combination of aspirin and an omega-3 fatty acid and the second agent.

The present invention provides a kit comprising:
a) a first pharmaceutical formulation comprising a compound of formula A, a compound of any one of formulae 1-49, a lipoxin compound, an oxylipin compound, or a combination of aspirin and an omega-3 fatty acid;
b) a second pharmaceutical formulation comprising a second agent suitable for modulating immune function, suppressing immune response, treating an autoimmune disease or autoimmune disorder, or treating a disease, sequela or pathological condition mediated by an activation of the immune system as mentioned above; and
c) instructions for the administration of the first and second pharmaceutical formulations.

In certain embodiments, the invention relates to a method for conducting a pharmaceutical business, by manufacturing a formulation of a compound of formula A, a compound of any one of formulae 1-49, a lipoxin compound, an oxylipin compound, or a combination of aspirin and an omega-3 fatty acid, or a kit as described herein, and marketing to healthcare providers the benefits of using the formulation or kit for modulating immune function, suppressing immune response, treating an autoimmune disease or autoimmune disorder, or treating a disease, sequela or pathological condition mediated by an activation of the immune system.

In certain embodiments, the invention relates to a method for conducting a pharmaceutical business, by providing a distribution network for selling a formulation of a compound of formula A, a compound of any one of formulae 1-49, a lipoxin compound, an oxylipin compound, or a combination of aspirin and an omega-3 fatty acid, or kit as described herein, and providing instruction material to patients or physicians for using the formulation for modulating immune function, suppressing immune response, treating an autoimmune disease or autoimmune disorder, or treating a disease, sequela or pathological condition mediated by an activation of the immune system.

In certain embodiments, the invention comprises a method for conducting a pharmaceutical business, by determining an appropriate formulation and dosage of a compound of formula A, a compound of any one of formulae 1-49, a lipoxin compound, an oxylipin compound, or a combination of aspirin and an omega-3 fatty acid for modulating immune function, suppressing immune response, treating an autoimmune disease or autoimmune disorder, or treating a disease, sequela or pathological condition mediated by an activation of the immune system, conducting therapeutic profiling of identified formulations for efficacy and toxicity in animals, and providing a distribution network for selling an identified preparation as having an acceptable therapeutic profile. In certain embodiments, the method further includes providing a sales group for marketing the preparation to healthcare providers.

In certain embodiments, the invention relates to a method for conducting a pharmaceutical business by determining an appropriate formulation and dosage of a compound of formula A, a compound of any one of formulae 1-49, a lipoxin compound, an oxylipin compound, or a combination of aspirin and an omega-3 fatty acid for modulating immune function, suppressing immune response, treating an autoimmune disease or autoimmune disorder, or treating a disease, sequela or pathological condition mediated by an activation of the immune system, and licensing, to a third party, the rights for further development and sale of the formulation.

### EXEMPLIFICATION

The biological activity of one or more of a compound of formula A, a compound of any one of formulae 1-49, a lipoxin compound, an oxylipin compound, or a combination of aspirin and an omega-3 fatty acid can be assessed using techniques well known in the art, such as those discussed below.

### Example 1

### Study of the Ex Vivo Activity of Compound X on CIA Rat Blood and Lymph Node Cells

Female Lewis rats were anesthetized with 3-5% isoflurane and arthritis was induced by intradermal injection of 0.3 mL (100 μL at three different sites) of an emulsion contagion 1.5 mg/mL of type II bovine collagen, CII (Elastin Products Inc., Cat# CM276), in incomplete Freund's adjuvant (IFA) at the base of the tail on day 0 and 6. Control rats were injected with an equal amount of IFA only.

The volume of both hind paws were measured using a water displacement plethysmometer (Ugo Basile, Biological Research Apparatus, Italy), and the onset of arthritis was indicated by increased paw volume, which appeared approximately on day 11 post injection. Both paw volumes and body weights were measured throughout the study every 2-3 days. Nineteen to twenty-one days after CII injection, the rats were euthanized with CO₂. Blood was collected by cardiac puncture and serum was saved.

The effects of Compound X,

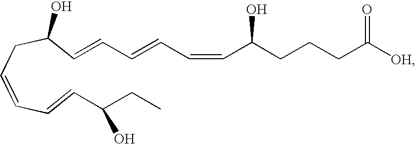

were measured under two different treatment regimens. In the first (prophylactic) treatment regimen, Compound X was administered i.v., twice daily, at 0.3 mg/kg from day 0 to day 6 or 10. For ex vivo studies, animals were euthanized on day 6 after the last injection and their inguinal lymph nodes were harvested. In the second (therapeutic) treatment regimen, Compound X was administered i.v., twice daily, at 0.3 mg/kg from day 8 (when pro-inflammatory markers are highly up-regulated) to day 16 or 19. For ex vivo studies, animals were euthanized on day 16 and inguinal lymph nodes were harvested. Venous blood samples were collected during the course of treatment. For CD3 activation, 96-well flat bottom plates were coated with anti-CD3 mAb (eBioscience, clone G4.18) 1 μg/mL. Plates were stored overnight at 4° C., and were subsequently rinsed with PBS once before use. Bovine Type II collagen for tissue culture was purchased from Chondrex (Cat# 2022) and was reconstituted according to the manufacturer's instructions. All data were processed and analyzed by t-test using GraphPad Prism software.

After the animals were euthanized, both hind paws and knees were removed, hind paws were weighed, and the paws and knees were placed in formalin. Following 1-2 days in fixative and 4-5 days in decalcifier, the ankle joints were cut in half longitudinally, and the knees were cut in half in the frontal plane. The joints were then processed, embedded, sectioned and stained with toluidine blue. Collagen arthritic ankles and knees were given scores of 0-5 for inflammation, pannus formation and bone resorption according to the following criteria:

### Knee Inflammation

### 0 Normal

1 Minimal infiltration of inflammatory cells in periarticular tissue
2 Mild infiltration
3 Moderate infiltration with moderate edema
4 Marked infiltration with marked edema
5 Severe infiltration with severe edema

### Knee Pannus

### 0 Normal

1 Minimal infiltration of pannus in cartilage and subchondral bone
2 Mild infiltration (extends over up to ¼ of surface or subchondral area of tibia or femur)
3 Moderate infiltration (extends over >¼ but <½ of surface or subchondral area of tibia or femur)
4 Marked infiltration (extends over ½ to ¾ of tibial or femoral surface)
5 Severe infiltration (covers >¾ of surface)

### Cartilage Damage (Knee, Emphasis on Femoral Condyles)

### 0 Normal

1 Minimal: minimal to mild loss of toluidine blue staining with no obvious chondrocyte loss or collagen disruption
2 Mild: mild loss of toluidine blue staining with focal mild (superficial) chondrocyte loss and/or collagen disruption
3 Moderate: moderate loss of toluidine blue staining with multifocal to diffuse moderate (depth to middle zone) chondrocyte loss and/or collagen disruption
4 Marked: marked loss of toluidine blue staining with multifocal to diffuse marked (depth to deep zone) chondrocyte loss and/or collagen disruption
5 Severe: severe diffuse loss of toluidine blue staining with multifocal severe (depth to tide mark) chondrocyte loss and/or collagen disruption on both femur and tibia

### Bone Resorption (Knee)

### 0 Normal

1 Minimal: small areas of resorption, not readily apparent on low magnification, rare osteoclasts
2 Mild: more numerous areas of resorption, definite loss of subchondral bone involving ¼ of tibial or femoral surface (medial or lateral)
3 Moderate: obvious resorption of subchondral bone involving >¼ but <½ of tibial or femoral surface (medial or lateral)
4 Marked: obvious resorption of subchondral bone involving ≧½ but <¾ of tibial or femoral surface (medial or lateral)
5 Severe: distortion of entire joint due to destruction involving >¾ of tibial or femoral surface (medial or lateral)

FIG. 1 shows that Compound X inhibited ex vivo IFN-γ and TNFα production in lymph node cells from collagen-induced arthritis (CIA) rats. Treatment regimen one was used (n=3). Animals were treated with Compound X from day 0 to day 6, were sacrificed on day 6, and freshly harvested lymph nodes were pressed through a nylon strainer (BD Falcon Cat# 352340) to obtain a single cell suspension. Cells were washed and resuspended in RPMI 1640/10% FCS at 4×10⁶/mL. In U-bottom 96-well plates, 100 μL or 4×10⁵ cells and 100 μL of collagen at 25 or 50 μg/mL final concentrations were added. Plates were incubated in a 37° C., 5% CO₂ incubator overnight. Supernatants were harvested, and rat cytokines were measured with Bioplex (BioRad).

FIG. 2 shows that Compound X inhibited ex vivo collagen-induced IFN-γ production in lymph node cells from collagen-induced arthritis (CIA) rats using two different treatment regimens. In FIG. 2A, animals were given compound X from Day 0 to Day 6 (treatment regimen one). In FIG. 2B, animals were given compound X from day 8 to day 16 (treatment regimen two).

FIG. 3 shows the Compound X inhibited ex vivo anti-CD3 mAb-induced IL-17 production in lymph node cells from collagen-induced arthritis rats using two different treatment regimens. In FIG. 3A, animals were given compound X from Day 0 to Day 6 (treatment regimen one). In FIG. 3B, animals were given compound X from day 8 to day 16 (treatment regimen two). Freshly harvested lymph nodes were pressed through a nylon strainer (BD Falcon Cat# 352340) to obtain a single cell suspension. Cells were washed and resuspended in RPMI 1640/10% FCS at 4×10⁶/mL. To anti-CD3 coated plates were added 4×10⁵ cells per well and 100 μL RPMI 1640/10% FCS media. The plates were incubated in a 37° C., 5% CO₂ incubator for three days (FIG. 3A) or overnight (FIG. 3B). Rat IL-17 was measured with a kit from Millipore (Cat# RCYTO-18K-01) following the manufacturer's instructions.

FIG. 4 shows that Compound X inhibited ex vivo LPS-stimulated cytokines in whole blood from CIA rats. To measure the effect of Compound X on blood cells, rat whole blood samples were taken on day 8, 10, 13, 16 and 20 (n=3-6). For the day 20 sample, vehicle or Compound X were given at 1 mg/kg orally, twice daily, starting at day 8. For the remainder of the samples, vehicle or Compound X were given at 0.3 mg/kg i.v., twice daily, starting at day 8. Blood was added to U-bottom 96 well plate (50 μL per well), diluted with 150 μL of RPMI 1640/10% FCS media, and challenged with 10 ng/mL LPS. After 4 hours in culture, supernatants were collected and cytokine levels measured by Bioplex.

These studies demonstrated that collagen- or CD3-induced IFNγ, TNFα and IL-17 secretion from cells isolated from draining lymph nodes was reduced by 60-90% in animals treated with Compound X compared to controls. Further, whole blood from CIA rats that were treated by Compound X had significantly lower cytokine production levels upon LPS-stimulation.

FIG. 5 shows that prophylactic dosing of Compound X inhibited arthritis in rats (n=9 for vehicle or treatment with Compound X; n=4 for non-arthritis control groups) with CIA. Specifically, twice daily i.v. dosing of Compound X at 0.3 mg/kg from days 0 to 10 showed a significant reduction of paw swelling (as measured by ankle diameter).

FIG. 6 shows that therapeutic dosing of Compound X inhibited arthritis in rats (n=9 for vehicle or treatment with Compound X; n=4 for non-arthritis control groups) with CIA. Specifically, twice daily i.v. dosing of Compound X at 0.3 mg/kg from days 8 to 19 showed a significant reduction in mean ankle joint volume.

FIG. 7 shows that therapeutic dosing of Compound X significantly reduced knee histopathology scores in rats (n=9 for vehicle or treatment with Compound X; n=4 for non-arthritis control groups) with CIA. Specifically, twice daily i.v. dosing of Compound X at 0.3 mg/kg from days 8 to 19 showed a significant reduction in inflammation, pannus, cartilage damage, and bone resorption as determined by knee histopathology scoring.

FIG. 8 shows that therapeutic dosing of Compound X protected bone resorption and joint damage in rats (n=9 for vehicle or treatment with Compound X; n=4 for non-arthritis control groups) with CIA.

### Example 2

### Study of the Ex Vivo Activity of Compound X on Mouse Spleen Cells

BALB/c female mice (n=5), 6-8 weeks old, were given either i.v. injection of Compound X or vehicle once a day for 5 days or a single i.v. injection of Compound X or vehicle. Thirty minutes after the last injection, spleens were removed under sterile conditions, and a single cell suspension was made by pressing through a nylon strainer (BD Falcon Cat# 352340). Cells were spun at 1500 rpm for 10 minutes, and the liquid was aspirated. RBC was lysed by adding 3 mL of ACK solution (Lonza Cat# 10-458E) for 5 minutes. The tube was filled with RPMI media and spun. Cells were resuspended in RPMI/10% FCS. To plates pre-coated with anti-CD3 (per the procedure described below) were added 4×10⁵ cells per well. Supernatants were harvested after 18 hours of culture. Cytokine levels were measured by Bioplex (Biorad), according to manufacturer's instructions.

For CD3 stimulation, anti-mouse CD3 mAb (BD Pharmingen clone 145-2C11) was used at 0.2 μg/mL in PBS to coat flat bottom 96-well plate at 4° C. overnight. Plates were rinsed twice with PBS before use.

FIG. 9 shows that Compound X inhibits cytokine release of CD3-stimulated mouse splenocytes. To measure this effect, mice were given daily i.v. injections of 0.3 mg/kg of Compound X for five days. Spleen cells were then harvested and stimulated with anti-CD3 antibody in vitro overnight.

FIG. 10 shows that acute treatment of Compound X in vivo resulted in reduction of CD3-induced cytokine release. Mice were given a single i.v. injection of 0.03 mg/kg Compound X 30 minutes before spleen cells were harvested. T cells were isolated by magnetic beads, and the purity of T cells was >95% as measured by flow cytometry. Spleen cells or purified T cells were stimulated with 0.2 μg/mL anti-CD3 overnight, and cytokine levels were measured by Bioplex.

The ability of in vivo treatment with Compound X to inhibit anti-CD3 stimulated cytokine production was measured by treating mice (n=5) with a single i.v. injection of 0.03 mg/kg of Compound X thirty minutes before spleens were harvested. Spleen cells were stimulated with anti-CD3 mAb overnight, and cytokine levels were measured by Bioplex. The resulting percent inhibition of cytokine production by total spleen cells as compared with vehicle treated animals is shown in Table 2. These studies indicate that Compound X, when dosed to naïve animals, inhibited ex vivo CD3-stimulated cytokine production in splenocytes by approximately 35-60%.

TABLE 2

| | % Inhibition |
|---|---|
| Il-2 | 62.6 |
| Il-4 | 64.9 |
| IL-6 | 55.2 |
| IL-13 | 68.3 |
| INFγ | 78.5 |
| MIP1α | 42.7 |
| MIP1β | 46.1 |
| RANTES | 37.2 |
| TNFα | 50.7 |

### Example 3

### Study of the In Vitro Activity of Compound X on Mouse Spleen Cells

BALB/c female mice, 6-8 weeks old, were euthanized and spleens were removed under sterile conditions. Spleens were gently pressed through a nylon strainer. Spleen cells and remaining connective tissue mass were incubated in a 6-well plate with 4 mL of Growth Media (RPMI 1640 supplemented with 10% FCS). Compound X was added to give a final concentration of 10 nM or 1000 nM. Control groups were spleen cells without addition of Compound X. All groups were in triplicates (3 animals per group). Plates were cultured in a 37° C., 5% CO₂ incubator. Once a day, 1 mL of culture media was removed from each well and replaced with 1 mL fresh growth media containing the same concentration of Compound X or fresh media only. Culture was continued for 5 days. All cells were then removed from the plates. Cells were passed through a nylon strainer to remove any aggregates, washed, counted and adjusted to 2.5×10⁶/mL in growth media. To anti-CD3 coated plates (coated per the procedure described below) were added 200 μL, or 5×10⁵ cells per well. Cells were cultured in a 37° C., 5% CO₂ incubator overnight. Supernatants were harvested, and cytokine levels were measured by Bioplex.

For CD3 stimulation, anti-mouse CD3 mAb (BD Pharmingen clone 145-2C11) was used at 0.2 μg/mL in PBS to coat flat bottom 96-well plate at 4° C. overnight. Plates were rinsed twice with PBS before use.

FIG. 11 shows that in vitro treatment with Compound X inhibited CD3-induced cytokine production of spleen cells.

### Example 4

### Delayed-Type-Hypersensitivity Model

Ear-swelling (swelling of the skin on the pinnae) was the endpoint of this model. This study was carried out over 6 to 7 days, with events occurring as described below.

Day 0 and/or Day 1: Sensitization

Mice (female BALB/c; n=10/group) were immunized with 0.5% of DNFB dissolved in acetone/olive oil. Sensitization was done with the contact allergen, wherein 20 μL of the solution was placed on the footpads of the animals.

### Day 5: Challenge & Dosing

Animals were dosed i.v. with the compounds 15 minutes prior challenge. The mice were challenged at day 5. 10 μL of a 0.8% DNFB solution was topically applied externally on the right ear of the animal. As control, the left ear was treated the same way with vehicle alone (acetone/olive oil).

### Day 6 and day 7: Measurement & Dosing

Animals were anesthetized with isoflurane and the ear swelling was measured within 24 to 48 hours after challenge using a micrometer. The micrometer calipers were closed around the top portion of the each external ear until resistance from the ear was felt. The compounds (controls and test) were administered once daily on days 6 and 7 until the completion of the study.

FIG. 12a shows that Compound X inhibited inflammation in murine DNFB-induced DTH model in a dose-dependent manner. Specifically, Compound X dose-dependently reduced tissue swelling in mice (n=10) with a maximal efficacy of 38% inhibition at 30 μg/kg when administered i.v., 15 minutes before challenge. Increment in ear thickness was measured 24 hours after challenge (day 6). The data shown in FIG. 12a is an average of 2-4 experiments. FIG. 12b shows that Compound X and dexamethasone treatment resulted in comparable levels of inhibition of the DTH response in mice (n=10; N=number of studies). Dexamethasone was administered 60 minutes prior to the DNFB challenge.

FIG. 13 shows that treatment with Compound X using two different regimens resulted in comparable and significant reduction of the DNFB-DTH response. Specifically, mice were treated once daily with an i.p. injection of 0.03 mg/kg of Compound X from day 0 to day 5, or with a single i.p. injection of 0.03 mg/kg of Compound X on day 5.

### Example 5

### Effects of Compounds in 11 Day Male DBA/1J Mouse Established Type II Collagen Arthritis

Male DBA/1J mice (7-9 weeks old on arrival; at least 7 weeks old at time of first immunization) were housed 5 per cage and were acclimated for enough days after arrival such that all animals were at least 7 weeks old at start of study.

Mice were anesthetized with Isoflurane and given intradermal collagen (2 mg/ml) injections at the base of the tail in a volume of 150 μl (D0 and D21). On days 21-35, onset of arthritis occurred and mice were randomized into treatment groups. Randomization into each group was done after swelling was obviously established in at least one paw, and attempts were made to assure approximately equal mean scores across the groups at time of enrollment. Treatment was initiated after enrollment and continued every day for a total of 10 days as outlined in Table 3. During the ten days of treatment, clinical scores were given for each of the paws (right front, left front, right rear, and left rear) according to the scoring methods provided below.

### Clinical Scoring Criteria for Fore and Hind Paws

0 normal
1 1 hind or fore paw joint affected or minimal diffuse erythema and swelling
2 2 hind or fore paw joints affected or mild diffuse erythema and swelling
3 3 hind or fore paw joints affected or moderate diffuse erythema and swelling
4 Marked diffuse erythema and swelling, or 4 digit joints affected
5 Severe diffuse erythema and severe swelling entire paw, unable to flex digits

All dose solutions were prepared to deliver 10 ml/kg (0.3 ml/30 g mouse). On day 11 of arthritis, animals were euthanized, and both fore and hind limbs with knees were removed, placed into formalin and then processed for microscopy. Following 1-2 days in fixative and then 4-5 days in decalcifier, the joints were processed, embedded, sectioned and stained with toluidine blue. (Only fore and hind paws and knees were initially processed—6 joints/mouse.)

Histopathologic Scoring Methods for Mouse Joints with Type II Collagen Arthritis

When scoring paws or ankles from mice with lesions of type II collagen arthritis, severity of changes as well as number of individual joints affected must be considered. When only 1-3 joints of the paws or ankles out of a possibility of numerous metacarpal/metatarsal/digit or tarsal/tibiotarsal joints were affected, an arbitrary assignment of a maximum score of 1, 2 or 3 from the scoring scale below was given depending on severity of changes. If more than 3 joints were involved, the full scoring scale below was applied only to the most severely affected/majority of joints.

### Pannus

### 0 Normal

1 Minimal infiltration of pannus in cartilage and subchondral bone
2 Mild infiltration with marginal zone destruction of hard tissue in affected joints
3 Moderate infiltration with moderate hard tissue destruction in affected joints
4 Marked infiltration with marked destruction of joint architecture, most joints
5 Severe infiltration associated with total or near total destruction of joint architecture, affects all joints

### Bone Resorption Scores

0 Normal
1 Minimal—small areas of resorption, not readily apparent on low magnification, rare osteoclasts in affected joints
2 Mild—more numerous areas of resorption, not readily apparent on low magnification, osteoclasts more numerous in affected joints
3 Moderate—obvious resorption of medullary trabecular and cortical bone without full thickness defects in cortex, loss of some medullary trabeculae, lesion apparent on low magnification, osteoclasts more numerous in affected joints
4 Marked—Full thickness defects in cortical bone, often with distortion of profile of remaining cortical surface, marked loss of medullary bone, numerous osteoclasts, affects most joints
Severe—Full thickness defects in cortical bone and destruction of joint architecture of all joints

### Statistical Analysis

Histologic parameters (mean±SE) for each group were analyzed for differences using the Chi Square Test and one way ANOVA. Significance was set at p≦0.05.

TABLE 3

| | | | | | Dose level | |
|---|---|---|---|---|---|---|
| ID | N | Treatment | Route | Regimen | (μg/kg) | |
| Grp 1 | 5 | Normal | iv | qd | 0 | |
| | | Control | | | | |
| Grp 2 | 15 or 16 | Disease | iv | qd | 0 | |
| | | Control | | | | |
| | | (vehicle) | | | | |
| Grp 3 | 15 or 16 | Cmpd X | iv | qd | 0.5 | |
| Grp 4 | 15 or 16 | Cmpd X | iv | qd | 5 | |
| Grp 5 | 15 or 16 | Cmpd X | iv | qd | 50 | |
| Grp 6 | 15 or 16 | Dexa- | iv | qd | 0.2 | mg/kg |
| | | methasone | | | | |
| Grp 7 | 15 or 16 | Disease | iv | bid | 0 | |
| | | control | | | | |
| | | (vehicle) | | | | |
| Grp 8 | 15 or 16 | Cmpd X | iv | bid | 5 | |

FIG. 14 shows the effects of Compound X on bone damage as was determined by histologic scoring in joints of mice (n=15) with established Type II collagen arthritis. In particular, twice daily dosing of 5 μg/kg showed a significant decrease in the score of bone damage as compared to vehicle control.

FIG. 15 shows the effects of Compound X on a) arthritis as was determined by clinical scoring in the paws of mice (n=16) with established Type II collagen arthritis, and b) pannus formation and bone loss as determined by histologic scoring in mice (n=16) with established Type II collagen arthritis. In particular, once daily i.v. dosing of Compound X at both 0.5 and 5.0 μg/kg inhibited clinical symptoms of arthritis as compared to arthritic control. Furthermore, once daily i.v. dosing of Compound X at both 0.5 and 5.0 μg/kg reduced pannus formation and bone loss as compared to vehicle control.

### Incorporation by Reference

All publications and patents mentioned herein are hereby incorporated by reference in their entirety as if each individual publication or patent was specifically and individually indicated to be incorporated by reference. In particular, compounds of formula A or formulae 1-49 disclosed in WO 2005/105025, WO 2006/078457, WO 2007/041440, US 2003/0191184, WO 2004/014835, WO 2004/078143, U.S. Pat. No. 6,670,396, US 2003/0236423, and US 2005/0228047, lipoxin compounds disclosed in US 2002/0107289, US 2004/0019110, US 2006/0009521, US 2005/0203184, and US 2005/0113443, oxylipin compounds disclosed in WO2006/055965, WO 2007/090162, and WO 2008/103753, derivatives and/or analogs of eicosapentaenoic acid or docosahexaenoic acid disclosed in WO 2005/089744, US 2004/0044050, US 2004/0116408 and US 2005/0261255, and aspirin-triggered lipid mediators disclosed in U.S. Pat. No. 7,053,230 are incorporated by reference as suitable for use in compositions and methods of the present invention. In case of conflict of structures or naming of compounds between the present application and the referenced patent publications listed above, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of inhibiting immune function in a patient comprising administering to said patient a compound of formula A, a compound of any one of formulae 1-49, a lipoxin compound, an oxylipin compound, a prodrug of any of the foregoing, or a pharmaceutically acceptable salt of any of the foregoing.

2. A method of suppressing an immune response in a patient comprising administering to said patient a compound of formula A, a compound of any one of formulae 1-49, a lipoxin compound, an oxylipin compound, a prodrug of any of the foregoing, or a pharmaceutically acceptable salt of any of the foregoing.

3. A method of treating an autoimmune disease or an autoimmune disorder in a patient comprising administering to said patient a compound of formula A, a compound of any one of formulae 1-49, a lipoxin compound, an oxylipin compound, a prodrug of any of the foregoing, or a pharmaceutically acceptable salt of any of the foregoing.

4. A method of treating a disease, sequela or pathological condition mediated by an activation of the immune system in a patient comprising administering to said patient a compound of formula A, a compound of any one of formulae 1-49, a lipoxin compound, an oxylipin compound, a prodrug of any of the foregoing, or a pharmaceutically acceptable salt of any of the foregoing.

5. The method according to any one of claims 1 to 4, wherein the compound of formula A, compound of any one of formulae 1-49, lipoxin compound, or oxylipin compound is selected from a compound of any one of Formulae 1 to 132.

6. A method of inhibiting immune function in a patient, comprising administering to said patient aspirin and an omega-3 fatty acid.

7. A method of suppressing an immune response in a patient, comprising administering to said patient aspirin and an omega-3 fatty acid.

8. A method of treating an autoimmune disease or an autoimmune disorder in a patient, comprising administering to said patient aspirin and an omega-3 fatty acid.

9. A method of treating a disease, sequela or pathological condition mediated by an activation of the immune system in a patient, comprising administering to said patient aspirin and an omega-3 fatty acid.
